(19) United States
(12) Patent Application Publication
(10) Pub. No.: US 2023/0287417 A1
(43) Pub. Date: Sep. 14, 2023

(54) MUCOPENETRATING FORMULATIONS

(71) Applicant: Vivtex Corporation, North Cambridge, MA (US)

(72) Inventor: Thomas von Erlach, North Cambridge, MA (US)

(73) Assignee: Vivtex Corporation, North Cambridge, MA (US)

(21) Appl. No.: 18/014,707

(22) PCT Filed: Jul. 2, 2021
(86) PCT No.: PCT/US2021/040244

§ 371 Date: Jan. 5, 2023

Related U.S. Application Data
(60) Provisional application No. 63/048,617, filed on Jul. 6, 2020.

(51) Int. Cl. C12N 15/113; A61K 47/26

(52) U.S. Cl. CPC C12N 15/113; A61K 47/26; C12N 2310/11; C12N 2310/3515; C12N 2320/32; C12N 2320/31

(57) ABSTRACT

Disclosed herein are compositions and kits comprising a mucopenetrating substance, a therapeutic nucleic acid, and a cationic polymer in an amount sufficient to charge neutralize the therapeutic nucleic acid. Also provided herein are methods of using and producing the compositions and kits.

### RELATED APPLICATION

This application claims the benefit under 35 U.S.C. § 119(e) of U.S. provisional application No. 63/048,617, filed Jul. 6, 2020 which is incorporated by reference herein in its entirety.

### BACKGROUND

Delivery of a therapeutic molecule to the gastrointestinal tract is referred to as enteric delivery, which may include oral delivery, gastric delivery, or rectal delivery. Therapeutic molecules for enteric delivery are formulated with the intention of achieving a high level of absorption of the therapeutic molecule from the intestine, through the intestinal wall, and into circulating blood to achieve systemic delivery. Therapeutic molecules are usually absorbed from the intestine by passive transfer, which includes diffusion of a molecule through the lipid cell membrane of the epithelial cells lining the inside of intestines. Factors taken into consideration when formulating therapeutic molecules for enteric delivery (e.g., oral, gastric, or rectal delivery) include ionization and lipid solubility of the molecule, gastrointestinal motility, splanchnic blood flow, and molecule size.

### SUMMARY

Provided herein are compositions comprising therapeutic nucleic acids that are formulated to traverse the mucus layer covering the epithelial cell lining of the gastrointestinal (GI) tract and, in some embodiments, to traverse the epithelial cell lining. The present disclosure is based, at least in part, on experimental data demonstrating that the mucus layer forms a barrier that prevents charged molecules (such as therapeutic nucleic acids) from traversing the GI tract lining. Surprisingly, the data provided herein shows that where conventional permeability enhancers e.g., fatty acids, fail, the combination of molecular charge neutralization and mucopenetrating substance(s) succeeds. The formulations provided herein permit efficient and effective delivery of therapeutic nucleic acids and other closely related charged compounds through the mucus layer and lining of the GI tract.

Some aspects of the present disclosure provide compositions comprising a mucopenetrating substance, a therapeutic nucleic acid, and a cationic polymer in an amount sufficient to charge neutralize the therapeutic nucleic acid.

Other aspects of the present disclosure provide cells comprising a composition that includes a mucopenetrating substance, a therapeutic nucleic acid, and a cationic polymer in an amount sufficient to charge neutralize the therapeutic nucleic acid.

Further aspects of the present disclosure provide complexes produced by combining a mucopenetrating substance with a therapeutic nucleic acid, and a cationic polymer in an amount sufficient to charge neutralize the therapeutic nucleic acid.

Yet other aspects of the present disclosure provide methods comprising delivering to a subject a mucopenetrating substance, a therapeutic nucleic acid, and a cationic polymer in an amount sufficient to charge neutralize the therapeutic nucleic acid.

Still other aspects of the present disclosure provide methods for decreasing gene expression in a subject, comprising delivering to a mucosal surface of a subject a composition described herein, in an effective amount to decrease gene expression in a cell in a local region of the mucosal surface.

Some aspects of the present disclosure provide methods for synergistically decreasing gene expression in a subject, comprising delivering to a mucosal surface of a subject a C10 fatty acid and a composition as described herein, in an effective amount to synergistically decrease gene expression in a cell in a local region of the mucosal surface, optionally wherein the composition further comprises the C10 fatty acid.

In some embodiments, a mucopenetrating substance comprises a non-ionic emulsifier. A mucopenetrating substance, in some embodiments, has mucolytic activity and/or mucotransport activity.

In some embodiments, a therapeutic nucleic acid, and a cationic polymer form a complex through ionic interactions. The complex, in some embodiments, further comprises the mucopenetrating substance.

In some embodiments, a cationic polymer is a linear polymer. In some embodiments, a cationic polymer is a branched polymer.

In some embodiments, a cationic polymer comprises a cationic lipid. For example, a cationic polymer may be selected from the group consisting of: polyquaternium, PDMAEMA (poly(2-dimethylaminoethyl methacrylate), MADQUAT (poly(2-(trimethylamino)ethyl methacrylate)), polyallylamines, polyvinylamines, polyethylenimine, polylysines, cationic polyaminoacids, and cationic polysaccharides. In some embodiments, a cationic polymer is selected from the group consisting of: polyallylamines, polyethyleneimines, and polylysines.

In some embodiments, the cationic polymer is a polyethyleneimine, for example, a branched polyethyleneimine. In some embodiments, the polyethyleneimine has a molecular weight of about 5-30 kilodaltons (kDa) or about 10-25 kDa (e.g., about 10-20, about 10-15, about 15-25, about 15-20, about 10, about 15, about 20, or about 25 kDa).

In some embodiments, the cationic polymer is a polyallylamine. In some embodiments, the polyallylamine has a molecular weight of lower than about 50 kDa, or 50 kDa or lower (e.g., about 5-50, about 10-50, about 15-50, about 20-50, about 25-50, about 5-40, about 10-40, about 15-40, about 20-40, about 25-40, about 5-30, about 10-30, about 15-30, about 20-30, about 5-25, about 10-25, about 15-25, about 20-25, about 25-25, about 5, about 10, about 15, about 20, about 25, about 30, about 35, about 40, about 45, or about 50 kDa). In some embodiments, the polyallylamine has a molecular weight of lower than about 40 kDa, lower than about 30 kDa, or lower than about 20, kDa.

In some embodiments, the cationic polymer is a polylysine. In some embodiments, the polylysine has a molecular weight of about 10-55 kDa or about 15-50 kDa (e.g., about 20-50, about 25-50, about 30-50, about 35-50, about 40-50, about 45-50, about 15-40, about 20-40, about 25-40, about 30-40, about 35-40, about 15-30, about 20-30, about 25-30, about 15-25, about 20-25, about 15, about 20, about 25, about 30, about 35, about 40, about 45, or about 50 kDa).

In some embodiments, the concentration of cationic polymer in the composition is about 5-35 mg/ml or about 10-30 mg/ml (e.g., about 15-30, about 20-30, about 25-30, about 10-20, about 15-20, about 10-15, about 10, about 15, about 20, about 25, or about 20 mg/ml).

In some embodiments, a non-ionic emulsifier is selected from the group consisting of: polysorbates, poloxamers, polyoxylglycerides, macrogolglycerol ricinoleate, polyethylene monostearate, sorbitan monoesters and triesters, substituted polyethylene glycols, and derivative thereof. In some embodiments, the non-ionic emulsifier is caprylocaproyl polyoxyl-8 glyceride (LABRASOL®), polysorbate 40 (TWEEN® 40), polysorbate 80 (TWEEN® 80), macrogolglycerol ricinoleate (KOLLIPHOR® P188), or oleoyl polyoxyl-6 glyceride/oleoyl macrogol-6 glyceride)(LABRAFIL®).

In some embodiments, the concentration of non-ionic emulsifier in the composition is about 5-45 mg/ml or about 10-40 mg/ml (e.g., about 15-40, about 20-40, about 25-40, about 30-40, about 35-40, about 10-30, about 15-30, about 20-30, about 25-30, about 10-20, about 15-20, about 10-15, about 10, about 15, about 20, about 25, about 20, about 25, about 30, about 35, or about 40 mg/ml).

In some embodiments, a mucopenetrating substance is selected from the group consisting of: bromohexine, L-cysteine methylester, bromalein, ambroxol, guaifenesin, and N-acetyl L-cysteine, and dornase alfa. In some embodiments, a mucopenetrating substance is bromalein or decanoic acid.

In some embodiments, a mucopenetrating substance is bromalein and/or the non-ionic emulsifier is oleoyl polyoxyl-6 glyceride/oleoyl macrogol-6 glyceride)(LABRAFIL®).

In some embodiments, a nucleic acid is deoxyribonucleic acid (DNA) or ribonucleic acid (RNA). A nucleic acid may have, for example, a phosphorothioate backbone. In some embodiments, the nucleic acid is double-stranded or single-stranded. A nucleic acid, in some embodiments comprises a modification, optionally a chemical modification and/or a genetic modification. In some embodiments, the nucleic acid has a length of 10 to 50 nucleotides. In some embodiments, the nucleic acid is not a locked nucleic acid (LNA) or a peptide nucleic acid (PNA).

In some embodiments, a therapeutic nucleic acid is selected from the group consisting of antisense oligonucleotides and RNA interference molecules. For example, the RNA interference molecules may be selected from the group consisting of short-hairpin RNAs (shRNAs), small-interfering RNAs (siRNAs), and micro RNAs (mRNAs).

In some embodiments, a therapeutic nucleic acid is an antisense oligonucleotide (ASO).

In some embodiments, a therapeutic nucleic acid targets SMAD7 mRNA. For example, a therapeutic nucleic acid may be mongersen (GED-0301).

In some embodiments, a cationic polymer and a therapeutic nucleic acid are present at a ratio of at least 1:1, at least 5:1, or at least 10:1 cationic polymer:therapeutic nucleic acid.

In some embodiments, compositions comprise an antisense oligonucleotide (ASO), polyethylenimine (PEI), and optionally a mucopenetrating substance, wherein the PEI is present in an amount sufficient to charge neutralize the therapeutic nucleic acid.

In some embodiments, compositions comprise an antisense oligonucleotide (ASO) (e.g., mongersen (GED-0301)), poly(2-(trimethylamino)ethyl methacrylate) (MADQUAT), and optionally a mucopenetrating substance, wherein the MADQUAT is present in an amount sufficient to charge neutralize the therapeutic nucleic acid.

In some embodiments, a composition comprising a mucopenetrating substance, a therapeutic nucleic acid, and a cationic polymer is in a solution, is lyophilized, or is in the form of a tablet, optionally with an enteric coating.

In some embodiments, a composition is a pharmaceutical composition further comprising a pharmaceutically-acceptable excipient.

In some embodiments, a therapeutic nucleic acid is an engineered nucleic acid, optionally a recombinant nucleic acid or a synthetic nucleic acid.

In some embodiments, delivery of a mucopenetrating substance, a therapeutic nucleic acid, and a cationic polymer is to a mucosal surface of the subject (e.g., orally, gastrointestinal tract, rectal tissue, or vaginal tissue).

In some embodiments, gene expression in a subject is reduced by at least 20% relative to gene expression in a subject relative to gene expression in a subject who has not received the composition or has received a composition comprising the therapeutic nucleic acid without the cationic polymer and/or the mucopenetrating substance.

In some embodiments, a subject has a gastrointestinal disorder and/or has a compromised gastrointestinal barrier. For example, a gastrointestinal disorder may be an inflammatory bowel disorder. In some embodiments, the inflammatory bowel disorder is irritable bowel syndrome (IBS), ulcerative colitis, or Crohn's disease.

In some embodiments, transport of the therapeutic nucleic acid through the mucosal surface is at least 5-fold, at least 10-fold, at least 15-fold, or at least 20-fold higher than uptake of a therapeutic nucleic acid without the cationic polymer and/or the mucopenetrating substance.

The present disclosure, in some aspects, also provides multiple well plates, wherein wells of the plates comprise a receiver chamber underlying a permeable membrane onto which a mucus layer has been deposited.

Also provided herein, in some aspects, are methods for assessing mucotransport of a substance, comprising applying the substance to a well, and assessing transport of the substance through the mucus layer. In some embodiments, a substance comprise a mucopenetrating substance.

Other aspects of the present disclosure provide a composition comprising an antisense oligonucleotide (ASO), non-ionic emulsifier, and a cationic polymer, wherein the composition comprises the cationic polymer in an amount sufficient to charge neutralize the ASO.

In some embodiments, the cationic polymer is selected from polyallylamine (PALL), polylysine (PLL), and polyethyleneimine (PEI). For example, the cationic polymer may be PALL. In some embodiments, the PALL has a molecule weight of lower than 50 kilodaltons (kDa). For example, the PALL has a molecular weight of about 10-20 kDa, optionally about 15 kDa. As another example, the cationic polymer may be PLL. In some embodiments, the PLL has a molecule weight of about 15-50 kDa. As yet another example, the cationic polymer may be PEI. In some embodiments, the PEI has a molecule weight of about 10-25 kDa. In some embodiments, the cationic polymer is branched. In some embodiments, the concentration of the cationic polymer in the composition is about 10-30 mg/ml.

In some embodiments, the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188. In some embodiments, the concentration of the non-ionic emulsifier is about 10-40 mg/ml.

In some embodiments, the cationic polymer is PALL, optionally having a molecule weight of below 50 kDa, and the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

In some embodiments, the cationic polymer is PLL, optionally having a molecule weight of about 15-50 kDa, and the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

In some embodiments, the cationic polymer is PEI, optionally branched PEI, optionally having a molecule weight of about 10-25 kDa, and the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

Other aspects of the present disclosure provide a composition comprising a therapeutic nucleic acid, a non-ionic emulsifier, and a cationic polymer having a molecular weight of 50 kDa or lower, wherein the composition comprises the cationic polymer in an amount sufficient to charge neutralize the ASO. In some embodiments, the cationic polymer has a molecular weight of about 10-50 kDa, about 15-50 kDa, or about 10-25 kDa. In some embodiments, the therapeutic nucleic acid is an antisense oligonucleotide (ASO).

Yet other aspects of the present disclosure provide a composition comprising an ASO, non-ionic emulsifier, and a zwitterionic polymer. In some embodiments, the zwitterionic polymer is polyvinylpyrrolidine. In some embodiments, the polyvinylpyrrolidine has a molecular weight of about 50-100 kDa.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows apical and basal jejunum tissue uptake of various formulations consisting of cationic polymers at different concentrations complexed to FAM-Mongersen. Results are summarized as a heatmaps that shows fold change relative to non-formulated Mongersen. White color indicates fold changes higher than 4-fold.

FIG. 2 shows apical and basal jejunum tissue uptake of various formulations consisting of cationic polymers complexed to FAM-Mongersen combined with non-ionic emulsifiers at different concentrations. Results are summarized as a heatmaps that shows fold change relative to non-formulated Mongersen. White color indicates fold changes higher than 15-fold.

FIG. 3 shows apical and basal jejunum tissue uptake of various formulations consisting of cationic polymers complexed to FAM-Mongersen combined with mucolytic agents at different concentrations. Results are summarized as a heatmaps that shows fold change relative to non-formulated Mongersen. White color indicates fold changes higher than 20-fold.

FIGS. 4A-4B show Least Squares Means Plots, which show relative change in tissue permeability and apical tissue accumulation of FAM-Mongersen using different molecular weight branched polyethyleneimine polymers. The results are based on a statistical regression analysis using 6 different non-ionic emulsifiers combined with polyethyleneimine-Mongersen polyplex.

FIG. 5 shows apical and basal jejunum tissue uptake of various formulations consisting of cationic polymers complexed to FAM-Mongersen combined with additional excipients: Non-ionic emulsifiers (Kolliphor P188, Labrafil, Tween 40, Tween 80), mycolytic (bromalein) or permeability enhancer/mucodisruptor (decanoic acid). FAM fluorescence intensity was quantified by fluorescence microscopy analysis of tissue cross-sections. The results show fold change relative to non-formulated Mongersen.

FIG. 6 shows apical and basal jejunum tissue uptake of various formulations consisting of cationic polymers complexed to FAM-Mongersen combined with non-ionic emulsifiers including Kolliphor P188, Poloxamer 407, Labrafil, Tween 20, Tween 40, Tween 60, Tween 80. FAM fluorescence intensity was quantified by fluorescence microscopy analysis of tissue cross-sections. The results show fold change relative to non-formulated Mongersen.

FIG. 7 shows tissue uptake in esophagus, stomach, jejunum, colon and rectum of various formulations consisting of cationic polymers complexed to FAM-Mongersen combined with non-ionic emulsifiers (Labrasol and Tween 40). FAM fluorescence intensity was quantified by fluorescence microscopy analysis of tissue cross-sections. The results show fold change relative to non-formulated Mongersen in the corresponding tissue segment.

FIG. 8 shows tissue uptake of various formulations consisting of cationic polymers complexed to FAM-Mongersen combined with non-ionic emulsifiers (Labrasol and Tween 40) in jejunum. FAM fluorescence intensity was quantified by fluorescence microscopy analysis of tissue cross-sections of biopsy samples harvested from jejunum segments exposed to formulations in pigs in vivo. The results show fold change relative to non-formulated Mongersen in the corresponding tissue segment.

FIGS. 9A-9B show effect of charged surfactants on tissue uptake of formulations. FIG. 9A shows tissue uptake of various formulations consisting of cationic polymers complexed to FAM-Mongersen combined with non-ionic emulsifiers (Labrasol and Tween 40) in jejunum. FAM fluorescence intensity was quantified by fluorescence microscopy analysis of tissue cross-sections of biopsy samples harvested from jejunum segments exposed to formulations in pigs in vivo. FIG. 9B shows the data in FIG. 9A in the form of a heatmap as show fold change relative to non-formulated Mongersen in the corresponding tissue segment.

FIGS. 10A-10B show IHC analysis of the fluorescence signal of Fam labeled Mongersen in vivo in pigs in the jejunum, where the Mongersen is treated with various Mongersen-polyplex formulations. effect of charged surfactants on tissue uptake of formulations.

FIGS. 11A-11B shows uptake of FAM-Mongersen into apical (FIG. 11A) and basal (FIG. 11B) intestinal tissue.

FIGS. 12A-12B shows uptake of FAM-Mongersen into apical (FIG. 11A) and basal (FIG. 11B) intestinal tissue for various formulations with and without mucolytics.

FIG. 13 shows transport through native porcine mucus obtained from the jejunum of various FAM-Mongersen formulations. Microdiffusion is calculated by measured FAM fluorescence intensity in receiver chamber compared to the initial donor fluorescence intensity after 1 hour of incubation.

FIG. 14 shows apical jejunum tissue uptake of various formulations consisting of cationic polymers complexed to Cy5-siRNA combined with non-ionic emulsifiers.

FIG. 15 shows apical jejunum tissue uptake of various formulations consisting of cationic polymers complexed to Cy3 conjugated plasmid DNA combined with non-ionic emulsifiers.

FIG. 16 shows a Least Squares Means Plot of the relative change in apical tissue accumulation of FAM-Mongersen using different molecular weight branched polyethyleneimine polymers. The results are based on a statistical regression analysis using six (6) different non-ionic emulsifiers combined with polyethyleneimine-Mongersen polyplex.

FIG. 17 shows the average apical tissue accumulation of FAM-Mongersen using different molecular weight polyallylamine polymers combined with four (4) different non-ionic emulsifiers. Values are expressed as fold change compared to the non-formulated FAM-Mongersen control.

FIG. 18 shows the average apical tissue accumulation of FAM-Mongersen using different molecular weight polylysine polymers combined with four (4) different non-ionic emulsifiers. Values are expressed as fold change compared to the non-formulated FAM-Mongersen control.

FIG. 19 shows the average apical tissue accumulation of FAM-Mongersen using different concentrations of polyallyllamine 15 kDa and non-ionic emulsifiers Kolliphor P188 and TWEEN® 80. Results are summarized as a bar graph that shows fold change relative to Mongersen in PBS buffer.

### DETAILED DESCRIPTION

Mucus is a viscoelastic and adhesive gel that has evolved to protect the gastrointestinal (GI) tract, lung airways, vagina, eye, and other mucosal surfaces by rapidly trapping and removing foreign particles and hydrophobic molecules. See Lai S K et al. Adv Drug Deliv Rev. 2009; 61(2): 158-171, incorporated herein by reference. Mucus is composed primarily of crosslinked and entangled mucin fibers secreted by goblet cells and submucosal glands. Mucins are large molecules (e.g., 0.5-40 MDa in size) formed by the linking of numerous mucin monomers (e.g., 0.3-0.5 MDa in size), and are coated with proteoglycans. In addition to mucins, mucus gels are loaded with cells, bacteria, lipids, salts, proteins, macromolecules, and cellular debris. The various components work together to form a nanoscopically heterogeneous environment for particle transport. Mucus viscoelasticity is tightly regulated in healthy subjects by controlling the mucin to water secretion ratio, as well as by varying lipid, protein, and ion content. See Lai S K et al. 2009.

The limited permeability of drug delivery particles and many hydrophobic drugs through the mucus barrier leads to their rapid clearance from the delivery site, often preventing effective biomolecular and drug therapies at non-toxic dosages. A number of diseases could be treated more effectively and with fewer side effects if therapeutic substances could be more efficiently delivered to the underlying mucosal tissues in a controlled manner. See Lai S K et al. 2009.

To avoid the rapid mucus clearance mechanism and/or reach the underlying epithelia, therapeutic substances must quickly traverse at least the outermost layers of the mucus barrier (that is cleared most rapidly). Mucus layer thickness depends strongly on anatomical site, and can range from less than 1 micron up to several hundred microns. To penetrate mucus, therapeutic substances, such as therapeutic nucleic acids, must avoid adhesion to mucin fibers and be small enough to avoid significant steric inhibition by the dense fiber mesh. See Lai S K et al. 2009. Further, the heterogeneity of mucus (e.g., within an individual or relative to two individuals) introduces variation in the mucopenetrability of therapeutic nucleic acids.

Therapeutic nucleic acids are nucleic acids (or closely related compounds) used to treat disease. See Sridharan K and Gogtay N J Br J Clin Pharmacol. 2016 September; 82(3): 659-672, incorporated herein by reference. Although there are various types of therapeutic nucleic acids, they share a common mechanism of action that is mediated by sequence-specific recognition of endogenous nucleic acids through Watson-Crick base pairing. Their development as therapeutic substances has specific distinct requirements because they fall somewhere between small molecules and biologics. Therapeutic nucleic acids are charged substances with physicochemical properties different from small molecule drugs and can be unstable in a biological environment. Further, nucleic acids typically have to be delivered to the correct intracellular compartment to have a therapeutic benefit. See Sridharan K and Gogtay N J 2016.

Both antisense oligonucleotides (ASOs) and aptamers are being explored as therapeutic nucleic acids. ASOs are single, short-stranded sequences (e.g., 8-50 base pairs in length) that bind to a target mRNA by means of standard Watson-Crick base pairing. After an ASO binds with the mRNA to form a target complex, either the target complex will be degraded by endogenous cellular RNase H or a functional blockade of mRNA occurs due to steric hindrance. Aptamers are single-stranded synthetic DNA or RNA molecules (e.g., 56-120 nucleotides in length) that bind with high affinity to the nucleotides coding for proteins and thus serve as decoys. DNA aptamers are short single-stranded oligonucleotide sequences with very high affinity for the target nucleic acids through structural recognition. See Sridharan K and Gogtay N J 2016.

RNA interference (RNAi) molecules are also being explored as therapeutic nucleic acids. RNAi is a process by which RNA molecules with sequences complementary to a gene coding sequence induce degradation of the corresponding messenger RNAs (mRNAs) thus blocking the translation of mRNA into protein. Therapy with siRNA thus has great potential application for diseases caused by abnormal expression or mutation such as cancers, viral infections and genetic disorders as RNA interference can be experimentally triggered. siRNAs are ‘short’ double-stranded molecules (e.g., 21-23 nucleotides long) and generally can be chemically synthesized. siRNAs have the advantage over DNA oligonucleotides in that they are always delivered as duplexes, which are more stable. Two major issues with siRNAs relate to their off-target effects and delivery into the cell. See Sridharan K and Gogtay N J 2016.

Various strategies for delivering charged therapeutic nucleic acids, successfully are being evaluated. Among these strategies include the use of cationic polymers and cationic lipids. Cationic polymers (including co-polymers) are often used to induce DNA condensation because they form strongly charged complexes with the anionic phosphate groups located on the DNA backbone. The resulting complexes can protect nucleic acids from enzymatic degradation and facilitate cellular entry. Cationic lipids form cationic liposomes that electrostatically bind to anionic nucleic acids, forming complexes (lipoplexes) that are taken up into cells by endocytosis. See Sasaki Y et al. Colloid and Interface Science in Pharmaceutical Research and Development, 2014.

Despite the ongoing research involving various therapeutic delivery strategies, delivery of charged therapeutic nucleic acids, to the GI tract remains a challenge. The present disclosure provides, inter alia, compositions for effective delivery of charged therapeutic nucleic acids to the GI tract. In some embodiments, the compositions include a mucopenetrating substance, a therapeutic nucleic acid, and a cationic polymer in an amount sufficient to charge neutralize the therapeutic nucleic acid.

### Mucopenetrating Substances

Compositions of the present disclosure, in some embodiments, comprise a (at least one) mucopenetrating substance. A mucopenetrating substance is a substance that facilitates the transport (penetration) of a therapeutic molecule through a mucus layer. It should be understood that the effects of a mucopenetrating substance on a therapeutic molecule can be assessed relative to a control condition, such as delivery (e.g., across a mucus layer and/or into the GI tract) of the particular therapeutic molecule in the absence of a particular mucopenetrating substance. In some embodiments, a mucopenetrating substance facilitates the transport of a therapeutic molecule through both the mucus layer and an underlying epithelial layer, such as the epithelial lining of the GI tract. A mucopenetrating substance may, for example, be formulated with a therapeutic molecule such that it binds to the therapeutic molecule through covalent or non-covalent interactions. In some embodiments, a mucopenetrating substance associates with a therapeutic molecule through electrostatic interactions. In some embodiments, such as those in which the therapeutic molecule is a nucleic acid, the mucopenetrating substance intercalates the nucleic acid (e.g., inserts between base pairs of DNA—see, e.g., work by Leonard Lerman discussed in Nucleic Acids in Chemistry and Biology, 3^{rd} Ed. Blackburn G M et al., RSC Publishing, 2006).

The mucus layer can prevent a therapeutic molecule from penetrating through the mucus layer through several different mechanisms. For example, a therapeutic molecule may associate with (e.g., bind to through non-covalent interactions) chyme and/or mucin fibers of the mucus layer and be targeted for excretion (see, e.g., Lai S K et al. (Adv Drug Deliv Rev. 2009; 61(2): 158-171). Thus, a mucopenetrating substance of the present disclosure, in some embodiments, facilitates the transport of a therapeutic molecule through the mucus layer by inhibiting the association between the therapeutic molecule and the chyme and/or the mucin fibers of the mucus layer.

Other mechanisms by which mucopenetrating substances can facilitate the transport of a therapeutic molecule (e.g., a therapeutic nucleic acid) through the mucus layer and/or underlying epithelial lining include transient opening of tight junctions in the epithelial lining, disruption of lipid bilayer packing in the epithelial lining, and/or altering the fluidity of the intestinal epithelial lining.

In some embodiments, a mucopenetrating substance improves passive transport of a therapeutic molecule through a mucus layer and/or underlying epithelial lining. In some embodiments, a mucopenetrating substance improves active transport of a therapeutic molecule through a mucus layer and/or underlying epithelial lining.

In some embodiments, a mucopenetrating substance increase the rate at which a therapeutic molecule traverses a mucus layer and/or underlying epithelial layer. For example, the rate at which a therapeutic molecule traverses a mucus layer and/or underlying epithelial layer when formulated with a mucopenetrating substance may increase by at least 10%, at least 20%, at least 30%, at least 40%, or at least 50%, relative to the therapeutic molecule not formulated with the mucopenetrating substance. In some embodiments, the rate at which a therapeutic molecule traverses a mucus layer and/or underlying epithelial layer when formulated with a mucopenetrating substance increases by 10%-50%, 20%-50%, 30%-50%, 40%-50%, 10%-100%, 20%-100%, 30%-100%, 40%-100%, or 50%-100%. In some embodiments, the rate at which a therapeutic molecule traverses a mucus layer and/or underlying epithelial layer when formulated with a mucopenetrating substance increases by at least 100%, or at least 200%.

In some embodiments, a mucopenetrating substance increase the amount of a therapeutic molecule that traverses a mucus layer and/or underlying epithelial layer. For example, the amount of a therapeutic molecule that traverses a mucus layer and/or underlying epithelial layer when formulated with a mucopenetrating substance may increase by at least 10%, at least 20%, at least 30%, at least 40%, or at least 50%, relative to the therapeutic molecule not formulated with the mucopenetrating substance. In some embodiments, the amount of a therapeutic molecule that traverses a mucus layer and/or underlying epithelial layer when formulated with a mucopenetrating substance increases by 10%-50%, 20%-50%, 30%-50%, 40%-50%, 10%-100%, 20%-100%, 30%-100%, 40%-100%, or 50%-100%. In some embodiments, the amount of a therapeutic molecule traverses a mucus layer and/or underlying epithelial layer when formulated with a mucopenetrating substance increases by at least 100%, or at least 200%.

In some embodiments, a mucopenetrating substance decreases the clearance rate (excretion) of a therapeutic molecule from the mucus layer. For example, the clearance rate of a therapeutic molecule when formulated with a mucopenetrating substance may decrease by at least 10%, at least 20%, at least 30%, at least 40%, or at least 50%, relative to the therapeutic molecule not formulated with the mucopenetrating substance. In some embodiments, the clearance rate of a therapeutic molecule when formulated with a mucopenetrating substance decreases by 10%-50%, 20%-50%, 30%-50%, 40%-50%, 10%-100%, 20%-100%, 30%-100%, 40%-100%, or 50%-100%. In some embodiments, the clearance rate of a therapeutic molecule when formulated with a mucopenetrating substance decreases by at least 100%, or at least 200%.

Mucolytic Substances

Mucopenetrating substances, in some embodiments, have mucolytic activity. This type of mucopenetrating substance is referred to herein as a mucolytic substance. Mucolytic substances alter the physical properties (e.g., viscosity) of a mucus layer in a way that facilitates transport of a therapeutic agent through the mucus layer. In some embodiments, a mucolytic substance exhibits enzymatic activity. There are several classes of mucolytic substances, including classic mucolytics, peptide mucolytics, and nondestructive mucolytics.

Classic mucolytics depolymerize the mucin glycoprotein oligomers by hydrolyzing the disulfide bonds that link the mucin monomers. Classic mucolytics typically contain sulfhydryl groups. A non-limiting example of a classic mucolytics is N-acetyl L-cysteine (NAC).

Peptide mucolytics depolymerize the DNA polymer (dornase alfa) or the F-actin network (e.g., gelsolin, thymosin β4). A non-limiting examples of a peptide mucolytics is dornase alfa (PULMOZYME™).

Nondestructive mucolytics are substances that “loosen” the polyionic tangled network of mucin that is formed by charged oligosaccharide side chains. Examples of nondestructive mucolytics include, but are not limited to, low-molecular-weight dextran and heparin.

Non limiting examples of mucolytic substances that may be used as provided herein include bromohexine, L-cysteine methylester, bromalein, ambroxol, guaifenesin, and bromohexine.

### Non-Ionic Emulsifiers

The compositions of the present disclosure, in some embodiments, comprise a (at least one) non-ionic emulsifier. In some embodiments, a non-ionic emulsifier is also a mucopenetrating substance. An emulsifier is a substance that can stabilize an emulsion, which is a mixture of two or more liquids that are otherwise immiscible. Emulsifiers generally keep molecules from precipitating out of a solution by providing hydrophobic groups onto which hydrophobic areas of the molecules can associate, thus preventing them from associating with other molecules and forming larger particles that are likely to leave the solution. Emulsifiers also typically have hydrophilic groups which keep them soluble in aqueous solutions of moderate to high ionic concentrations. Emulsifiers, natural or synthetic, can be nonionic, anionic, cationic, or amphoteric.

Non-ionic emulsifiers have no overall charge. In some embodiments, a non-ionic emulsifier comprises a hydrophilic portion that includes free hydroxyl and oxyethylene groups and a lipophilic portion having long-chain hydrocarbons of fatty acids and fatty alcohols. Non-limiting examples of natural non-ionic emulsifiers include fatty acid alcohols (e.g., stearyl alcohol and cetyl alcohol), wool fat or wool wax and its derivatives, wool alcohols and cholesterol, and derivatives of other natural waxes (e.g., such as spermaceti and cetyl esters wax (synthetic spermaceti). Non-limiting examples of synthetic non-ionic emulsifiers include complex esters and ester-ethers, derived from polyols, alkylene oxides, fatty acids, and fatty alcohols.

Other non-limiting examples of non-ionic emulsifiers include caprylocaproyl polyoxyl-8 glyceride (LABRASOL®), polysorbate 40 (TWEEN® 40), macrogolglycerol ricinoleate (KOLLIPHOR® P188), or oleoyl polyoxyl-6 glyceride/oleoyl macrogol-6 glyceride (LABRAFIL®).

### Therapeutic Nucleic Acids

Therapeutic nucleic acids are nucleic acids (or closely related compounds) used to treat disease. See Sridharan K and Gogtay N J Br J Clin Pharmacol. 2016 September; 82(3): 659-672, incorporated herein by reference. Treatment herein refers to a reduction in the frequency or severity of at least one sign or symptom of a disease or disorder experienced by a subject. The compositions described herein are typically administered to a subject in an effective amount, that is, an amount capable of producing a desirable result. The desirable result will depend upon the active agent being administered. For example, an effective amount of a composition comprising a therapeutic molecule (e.g., a therapeutic nucleic acid) may be an amount of the composition that is capable of causing a desirable expression of a gene or reduction in the expression of a gene in a host organ, tissue, or cell. A therapeutically acceptable amount may be an amount that is capable of treating a disease, e.g., a disease of the GI tract. As is well known in the medical and veterinary arts, dosage for any one subject depends on many factors, including the subject's size, body surface area, age, the particular composition to be administered, the active ingredient(s) in the composition, time and route of administration, general health, and other drugs being administered concurrently.

A therapeutic nucleic acid may be single-stranded or double-stranded. In some embodiments, a therapeutic nucleic acid may comprise one or more segments that are single-stranded and one or more segments that are double-stranded.

A therapeutic nucleic acid may be a DNA (e.g., a DNA based on antisense oligonucleotides or DNA aptamers) or an RNA (e.g., microRNAs, short interfering RNAs, ribozymes, RNA decoys, and circular RNAs). In some embodiments, a therapeutic nucleic acid is a DNA-RNA hybrid, having both DNA and RNA segment(s). A therapeutic molecule, in some embodiments, comprises a backbone that is different than that of a DNA or RNA. For example, a therapeutic nucleic acid may have a phosphorothioate backbone.

In some embodiments, a therapeutic nucleic acid is an antisense oligonucleotide. As discussed above, an antisense oligonucleotide is a single, short-stranded sequence (e.g., 8-50 base pairs in length) that binds to a target mRNA by means of standard Watson-Crick base pairing. See, e.g., Rinaldi C and Wood M. Nature Reviews Neurology 2018; 14: 9-21). For example, mongersen (GED-0301) is an antisense oligonucleotide used to block the transcription of RNA encoding SMAD7 protein.

In some embodiments, a therapeutic nucleic acid is a RNA interference molecule (e.g., a short-hairpin RNA (shRNA), a small-interfering RNAs (siRNA), or a micro RNA (mRNA)). As discussed above, RNAi is a process by which RNA molecules with sequences complementary to a gene coding sequence induce degradation of the corresponding messenger RNAs (mRNAs) thus blocking the translation of mRNA into protein. See, e.g., Setten R et al. Nature Reviews Drug Discovery 2019; 18: 421-446.

In some embodiments, a therapeutic nucleic acid has a length of 10-100 nucleotides. For example, a therapeutic nucleic acid may have a length of 10-75, 10-50, 10-25, 15-100, 15-75, 15-50, 15-25, 20-100, 20-75, 20-50, 25-100, 25-75, or 25-50 nucleotides. In some embodiments, a therapeutic nucleic acid has a length of 10, 11, 12, 13, 14, 15, 16, 17, 18, 19, 20, 21, 22, 23, 24, 25, 26, 27, 28, 29, or 30 nucleotides. In some embodiments, a therapeutic nucleic acid is longer than 100 nucleotides.

A therapeutic nucleic acid may be an engineered nucleic acid, which includes synthetic and recombinant nucleic acids. Synthetic nucleic acids are nucleic acids that are made by chemical synthesis. Recombinant nucleic acids are nucleic acids that are made using recombinant technologies (e.g., genetic recombination, and by having an organism such a bacteria make the desired nucleic acid).

Therapeutic nucleic acids may be modified. Non-limiting examples of nucleic acid modifications include addition of one or more deoxyinosine, deoxyuridine, amino dU, 2-aminopurine, 5-bromodeoxycytidine, 5-bromodeoxyuridine, aminohexyl (aminolink), phosphate, thiol, hexaethylene glycol, thiophosphate, 5-iododeoxyuridine, and 5-methyldeoxycytidine. In some embodiments, modifications are made to the sugar phosphate backbone, e.g., a Phosphorothioate backbone or a 2′-O-methyl backbone. In some embodiments, a therapeutic nucleic acid is formulated as a salt (e.g., a sodium salt). A modified therapeutic nucleic acid may or may not be a locked nucleic acid (LNA) or a peptide nucleic acid (PNA). A modification of a therapeutic nucleic acid may also be a genetic modification (e.g., an amino acid substitution).

In some embodiments, a therapeutic nucleic acid is conjugated to a small molecule or a protein or peptide (e.g., to target a particular cell type or tissue, or a dye molecule for detection purposes). In some embodiments, a nucleic acid is encapsidated or engulfed by a lipid substance.

In some embodiments, a therapeutic nucleic acid modulates (increases or decreases) expression of a molecular target for an inflammatory bowel disorder (e.g., disease). For example, a therapeutic nucleic acid may target any one or more of the molecules described by Katsanos K H and Papadakis K A Gut Liver 2017; 11(4): 455-463, incorporated herein by reference. Non-limiting examples of molecule targets include apoptotic molecules (e.g., caspase-8), Toll-like receptors (e.g., TLR-4), macrophages (TGFβ, TNF-α, IFN-γ, cytokines [IL-6, IL-9, IL-12, IL-23]), dendritic cells, defensins, regulatory T cells, T effector cells (Th1, Th2, Th17), B cells, dendritic cells, Smad7, JAK inhibitors (e.g., tofacitinib), adhesion molecules (e.g., MAdCAM-1), anti-integrins (e.g., anti-α4β7), genes involved in innate mucosal defense and antigen presentation (NOD2, MDR1, PPAR-γ), redox-sensitive signaling pathways and proinflammatory transcription molecules, dendritic cells, adipocytes, fibroblasts, and myofibroblasts.

### Cationic Polymer

Data provided herein shows that charge neutralizing an otherwise negatively-charged therapeutic molecule (e.g., a therapeutic nucleic acid) by formulating it with a cationic polymer i.e., 10-25 kDa and a mucopenetrating substance facilitates transport of an effective amount of the therapeutic molecule through both the mucus layer and the epithelial lining of the GI tract. Cationic macromolecules (e.g., cationic polymers) are macromolecules that have an overall positive charge. The positive charges of the cationic macromolecules may be attributed to either the macromolecular backbone, side chains, or both the backbone and the side chains. In some embodiments, a cationic macromolecule is naturally occurring. In other embodiments, a cationic macromolecule is synthetic (not naturally occurring). In some embodiments, a cationic macromolecule is a cationic polymer. A polymer is a macromolecule composed of repeated subunits. The role of cationic polymers in drug delivery systems is discussed by Farshbaf et al. in Artificial Cells, Nanomedicine, and Biotechnology 2018; 46(8): 1872-1891 and Samal et al. in Chem Soc Rev. 2012; 41(21): 7147-94, each of which is incorporated herein by reference). In some embodiments, a cationic polymer is a cationic lipid.

Charge neutralization refers to a state in which the net electrical charge of particles, fibers, colloidal material, and/or polyelectrolytes in aqueous solution have been canceled by the adsorption of an equal number of opposite charges. This may be characterized as a Zeta potential measurement. For example, as provided herein, an otherwise negatively charged nucleic acid may be complexed with a cationic macromolecule is an amount sufficient to charge neutralize the nucleic acid. That is, the overall negative charge of the nucleic acid is canceled by the positive charges of the cationic macromolecule.

In some embodiments, a cationic macromolecule is linear. In other embodiments, a cationic macromolecule is branched (e.g., PAMAM dendrimers).

In some embodiments, a cationic polymer is a cationic gelatin. In some embodiments, a cationic polymer is a cationic chitosan (e.g., chitosan low MW, chitosan high MW, chitosan medium MW). In some embodiments, a cationic polymer is a cationic cellulose. In some embodiments, a cationic polymer is a cationic dextran. See, e.g., Farshbaf et al. 2018.

Non-limiting examples of cationic polymers include poly(2-N,N-dimethylaminoethylmethacrylate) PDMAEMA, poly-L-lysine (PLL), poly(ethyleneeimine) (PEI), poly(amidoamine) (PAMAM), chitosan (e.g., low, medium, or high MW), dL-Lysine monohydrochloride, polydiallyidimethyl ammonium, polyethylenimine (e.g., of 25,000 MW, or 800 MW), ply 2-ethyldimethylammoinoethylmethacrylate ethyl sulfate-co-1-vinylspyrolidone (having an average MW of 1,000,000, also referred to herein as MADQUAT), poly 2-dimethylaminoethylmethacrylate methylchloride, poly L-Lysine hydrobromide (e.g., MW 1000-5000, 15000-25000, 5-15000, and 30000-70000), PLKC, mPEGK-b-PLKC50, and PLCK-PEG5K-b-PLK50. Any one or more of the preceding cationic polymers may be combined with an otherwise negatively-charged therapeutic nucleic acid in an amount sufficient to charge neutralize the therapeutic nucleic acid.

### Complexes

Provided herein, in some embodiments, are complexes comprising a therapeutic molecule and a cationic macromolecule. A complex, in some embodiments, further comprise a (at least one) mucopenetrating substance. In some embodiments, a therapeutic molecule and a cationic macromolecule (and/or a mucopenetrating substance) form a complex through non-covalent interactions, such as ionic interactions.

In some embodiments, a therapeutic molecule (e.g., a therapeutic nucleic acid) as provided herein is complexed with a cationic macromolecule. In some embodiments, a therapeutic molecule (e.g., a therapeutic nucleic acid) and a cationic macromolecule (e.g., a cationic polymer) form a complex through ionic interactions. A therapeutic molecule (e.g., a therapeutic nucleic acid) and a cationic macromolecule (e.g., a cationic polymer) may also be formed through covalent interactions.

In some embodiments, a complex comprising a therapeutic molecule (e.g., a therapeutic nucleic acid) and a cationic macromolecule (e.g., a cationic polymer) also comprises a mucopenetrating substance as described herein. The mucopenetrating substance may exhibit mucolytic activity, for example. In some embodiments, a complex comprises a therapeutic molecule (e.g., a therapeutic nucleic acid), a cationic macromolecule (e.g., a cationic polymer), a mucopenetrating substance, and a non-ionic emulsifier.

In some embodiments, a complex is produced by combining a mucopenetrating substance with a therapeutic nucleic acid and a cationic polymer in an amount sufficient to charge neutralize the therapeutic nucleic acid.

### Compositions

It is to be understood that a composition as provided herein can comprise any therapeutic molecule, any cationic macromolecule, and any mucopenetrating substance described herein. In some embodiments, a composition comprises at least two mucopenetrating substances, for example, a mucolytic substance and at least one other mucopenetrating substance. In some embodiments, a composition comprises a therapeutic molecule, a cationic macromolecule, a mucopenetrating substance (e.g., a mucolytic substance), and a non-ionic emulsifier. Non-limiting examples of compositions contemplated herein are provided in Table 1.

TABLE 1
Non-limiting examples of therapeutic compositions for delivery to the GI tract

| | | Mucopenetrating Substance | | |
|---|---|---|---|---|
| Therapeutic | | | | Additional Agent |
| Nucleic Acid | Cationic Polymer | Mucolytic | Non-ionic Emulsifier | (optional) |

| | | | |
|---|---|---|---|
| Antisense | polyethylenimine | bromalein | oleoyl polyoxyl-6 |
| oligonucleotide | (PEI) | | glyceride/oleoyl |
| (ASO) | | | macrogol-6 glyceride |
| | | | (LABRAFIL ®) |
| ASO | MADQUAT | bromalein | oleoyl polyoxyl-6 |
| | | | glyceride/oleoyl |
| | | | macrogol-6 glyceride |
| | | | (LABRAFIL ®) |

In some embodiments, a composition comprises an antisense oligonucleotide (ASO), polyethylenimine (PEI), and a mucopenetrating substance, wherein the PEI is present in an amount sufficient to charge neutralize the therapeutic nucleic acid.

In some embodiments, a composition comprises an ASO, poly(2-(trimethylamino)ethyl methacrylate) (MADQUAT), and a mucopenetrating substance, wherein the MADQUAT is present in an amount sufficient to charge neutralize the therapeutic nucleic acid.

The concentration of therapeutic molecule in a composition may vary, depending on the particular molecule and the intended therapeutic effect. In some embodiments, the concentration of a therapeutic molecule is 0.0001-1000 mg/ml.

The concentration of cationic macromolecule in a composition may vary, depending on the amount required to charge neutralize the therapeutic molecule in the composition. In some embodiments, the concentration of a cationic macromolecule is 0.0001-1000 mg/ml.

The concentration of a mucopenetrating substance in a composition may vary. In some embodiments, the concentration of a mucopenetrating substance is 0.0001-1000 mg/ml.

The concentration of a non-ionic emulsifier in a composition may vary. In some embodiments, the concentration of a non-ionic emulsifier is 0.0001-1000 mg/ml.

The ratio of any two substances in a composition may vary. For example, the ratio of any two substances (e.g., cationic macromolecule and therapeutic molecule, cationic molecule and mucopenetrating substance, cationic macromolecule and non-ionic emulsifier, therapeutic molecule and mucopenetrating substance, therapeutic molecule and non-ionic emulsifier, or mucopenetrating substance and non-ionic emulsifier) may be at least 1:1 to at least 10:1 (e.g., at least 1:1, at least 2:1, at least 3:1, at least 4:1, at least 5:1, at least 6:1, at least 7:1, at least 8:1, at least 9:1, or at least 10:1).

In some embodiments, a composition comprises a cationic macromolecule (e.g., polymer) and a therapeutic molecule (e.g., nucleic acid) at a ratio sufficient to charge neutralize the therapeutic molecule. In some embodiments, a cationic macromolecule and the therapeutic molecule are present at a ratio of at least 1:1 (e.g., at least 1:1, at least 2:1, at least 3:1, at least 4:1, at least 5:1, or at least 10:1) cationic macromolecule:therapeutic molecule. In some embodiments, a cationic macromolecule and the therapeutic molecule are present at a ratio of more than 10:1 (e.g., at least 12:1, at least 15:1, at least 20:1, at least 50:1, or at least 100:1) cationic macromolecule:therapeutic molecule.

In some embodiments, a composition comprises a cationic macromolecule (e.g., polymer) a therapeutic molecule (e.g., nucleic acid), and a mucopenetrating substance. In some embodiments, the mucopenetrating substance and the therapeutic molecule and are present at a ratio of at least 1:1 (e.g., at least 1:1, at least 2:1, at least 3:1, at least 4:1, at least 5:1, or at least 10:1) mucopenetrating substance:therapeutic molecule. In some embodiments, a cationic macromolecule and the therapeutic molecule are present at a ratio of more than 10:1 (e.g., at least 12:1, at least 15:1, at least 20:1, at least 50:1, or at least 100:1 mucopenetrating substance:therapeutic molecule.

Compositions comprising therapeutic nucleic acids may be formulated, for example, as a solid or a liquid for oral, rectal, gastric, or vaginal delivery. In some embodiments, a composition is formulated as a solid tablet or a lyophilized powder. In some embodiments, a solid dosage form has a protective coating (e.g., an enteric coating). Non-limiting examples of protective coatings include methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, shellac, cellulose acetate trimellitate, sodium alginate, and zein. In some embodiments, a composition is formulated as a slow-release composition.

Composition herein may further comprise a pharmaceutically acceptable excipient (e.g., carrier, buffer, and/or salt, etc.). A molecule or other substance/agent is considered “pharmaceutically acceptable” if it is approved or approvable by a regulatory agency of the Federal government or a state government or listed in the U.S. Pharmacopeia or other generally recognized pharmacopeia for use in animals, including humans. An excipient may be any inert (inactive), non-toxic agent, administered in combination with a therapeutic molecule. Non-limiting examples of excipients include buffers (e.g., sterile saline), salts, carriers, preservatives, fillers, coloring agents.

### Cells and Tissues

Provided here are cells comprising any one of the compositions comprising therapeutic nucleic acids described herein. In some embodiments, the cell is a gastrointestinal tract cell. For example, a cell may be an intestinal epithelial cell that lines the surface of intestinal epithelium. A cell as provided herein may be an isolated cell, part of a tissue, or present in a subject (e.g., in the GI tract of a subject or model organism). In some embodiments, a cell is a human cell, a pig cell, or a rodent cell.

### Methods

Any of the compositions described herein may be delivered to a subject, for example, to treat a gastrointestinal disorder (e.g., disease). In some embodiments, the gastrointestinal disorder is an inflammatory bowel disorder. Non-limiting examples of inflammatory bowel disorders include irritable bowel syndrome (IBS), ulcerative colitis, and Crohn's disease.

A subject, in some embodiments, is a human subject. Other mammalian subjects are contemplated herein. For example, a subject may be a veterinary subject (e.g., cat, dog, horse, cow, sheep, pig, etc.).

The route of delivery may be oral, nasal, intravenous, subcutaneous, intramuscular, or intraperitoneal. Other routes of delivery are contemplated herein. In some embodiments, the route of delivery is oral, for example, a composition is formulated as a enteric-coated table. In some embodiments, a composition is delivered, directly or indirectly, to a mucosal surface of a subject (e.g., mucosal layer lining the GI tract).

The methods herein encompass delivery of a single composition or delivery of multiple composition, simultaneously or successively. For example, a method herein may include delivering a subject a composition comprising a therapeutic molecule and cationic polymer and also delivering to the subject a composition comprising a mucopenetrating substance and/or a non-ionic emulsifier.

In some embodiments, delivery of a composition comprising a therapeutic molecule, such as a therapeutic nucleic acid, results in a decrease in gene expression in a cell of the subject. Thus, a therapeutic nucleic acid may target a gene of interest and inhibit expression of that gene, for example, by binding to the gene or the mRNA encoded by the gene. Thus, in some embodiments, a method comprises delivering to a subject a composition comprising a mucopenetrating substance, a therapeutic nucleic acid, and a cationic polymer in an amount sufficient to charge neutralize the therapeutic nucleic acid, wherein delivery of the composition decreases gene expression in a cell of the subject, relative to baseline expression of the gene (not exposed to the therapeutic nucleic acid) or relative to expression of the gene following delivery of a control composition with the therapeutic nucleic acid but without the cationic macromolecule and/or mucopenetrating substance. In some embodiments, gene expression is decreased by at least 10%, at least 20%, at least 30%, at least 40%, or at least 50%, relative to a control.

Also provided herein are methods for synergistically decreasing gene expression in a subject, comprising delivering to a mucosal surface of a subject a C10 fatty acid and a composition comprising a mucopenetrating substance, a therapeutic nucleic acid, and a cationic polymer in an amount sufficient to charge neutralize the therapeutic nucleic acid, wherein delivery of the composition synergistically decreases gene expression in a cell of the subject. In some embodiments, the composition further comprises the C10 fatty acid. Synergy refers to the interaction or cooperation of two or more substances to produce a combined effect greater than the sum of their separate effects. In some embodiments, gene expression is decreased by at least 10%, at least 20%, at least 30%, at least 40%, or at least 50%, relative to a control.

### Devices

Computational oral physiologically-based pharmacokinetic (PBPK) models are used to predict oral bioavailability of therapeutic nucleic acids and formulations thereof. See Lin and Harve, Pharmaceutics. 2017 Sep. 26; 9(4). Pre-clinical methods to confirm or refute predictions based on PBPK models are of great significance as results obtained at the pre-clinical stage can help save resources at the clinical stage. Provided herein are preclinical assays and assay systems to assess the mucopenetrability and/or enteric absorption of therapeutic nucleic acids.

Some aspects of the present disclosure provide a multiple well plate, wherein each well of the plate comprises a receiver chamber underlying a permeable membrane onto which a mucus layer has been deposited.

Some aspects of the present disclosure provide methods for assessing mucotransport of a substance, comprising applying the substance to a well described herein, and assessing transport of the substance through the mucus layer.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever.

### Additional Embodiments

Additional embodiments of the present disclosure are encompassed by the following numbered paragraphs:

1. A composition comprising a mucopenetrating substance, a therapeutic nucleic acid, and a cationic polymer in an amount sufficient to charge neutralize the therapeutic nucleic acid.

2. A composition comprising an antisense oligonucleotide (ASO), polyethylenimine (PEI), and optionally a mucopenetrating substance, wherein the PEI is present in an amount sufficient to charge neutralize the therapeutic nucleic acid.

3. A composition comprising an antisense oligonucleotide (ASO), poly(2-(trimethylamino)ethyl methacrylate) (MADQUAT), and optionally a mucopenetrating substance, wherein the MADQUAT is present in an amount sufficient to charge neutralize the therapeutic nucleic acid.

4. A composition comprising an antisense oligonucleotide (ASO), non-ionic emulsifier, and a cationic polymer, wherein the composition comprises the cationic polymer in an amount sufficient to charge neutralize the ASO.

5. A composition comprising a therapeutic nucleic acid, a non-ionic emulsifier, and a cationic polymer having a molecular weight of 50 kDa or lower, wherein the composition comprises the cationic polymer in an amount sufficient to charge neutralize the ASO.

6. A composition comprising an antisense oligonucleotide (ASO), non-ionic emulsifier, and a zwitterionic polymer.

7. The composition of any one of the preceding numbered paragraphs, wherein the mucopenetrating substance is a non-ionic emulsifier and/or has mucolytic activity.

8. The composition of any one of the preceding numbered paragraphs, wherein the therapeutic nucleic acid and the cationic polymer form a complex through ionic interactions.

9. The composition of any one of the preceding numbered paragraphs, wherein the complex further comprises the mucopenetrating substance.

10. The composition of any one of the preceding numbered paragraphs, wherein the composition comprises at least two or at least three mucopenetrating substances.

11. The composition of any one of the preceding numbered paragraphs, wherein the cationic polymer is a linear polymer.

12. The composition of any one of the preceding numbered paragraphs, wherein the cationic polymer is a branched polymer.

13. The composition of any one of the preceding numbered paragraphs, wherein the cationic polymer comprises a cationic lipid.

14. The composition of any one of the preceding numbered paragraphs, wherein the cationic polymer is selected from the group consisting of: polyquaternium, PDMAEMA (poly(2-dimethylaminoethyl methacrylate), MADQUAT (poly(2-(trimethylamino)ethyl methacrylate)), polyallylamines, polyvinylamines, polyethylenimine, polylysines, cationic polyaminoacids, and cationic polysaccharides.

15. The composition of any one of the preceding numbered paragraphs, wherein the cationic polymer is selected from the group consisting of: polyallylamines, polyethyleneimines, and polylysines.

16. The composition of any one of the preceding numbered paragraphs, wherein the cationic polymer is a polyethyleneimine.

17. The composition of any one of the preceding numbered paragraphs, wherein the polyethyleneimine is a branched polyethyleneimine.

18. The composition of any one of the preceding numbered paragraphs, wherein the polyethyleneimine has a molecular weight of about 10-25 kilodaltons (kDa).

19. The composition of any one of the preceding numbered paragraphs, wherein the cationic polymer is a polyallylamine.

20. The composition of any one of the preceding numbered paragraphs, wherein the polyallylamine has a molecular weight of lower than 50 kDa.

21. The composition of any one of the preceding numbered paragraphs, wherein the cationic polymer is a polylysine.

22. The composition of any one of the preceding numbered paragraphs, wherein the polylysine has a molecular weight of about 15-50 kDa.

23. The composition of any one of the preceding numbered paragraphs, wherein the concentration of cationic polymer in the composition is about 10-30 mg/ml.

24. The composition of any one of the preceding numbered paragraphs, wherein the non-ionic emulsifier is selected from the group consisting of: polysorbates, poloxamers, polyoxylglycerides, macrogolglycerol ricinoleate, polyethylene monostearate, sorbitan monoesters and triesters, substituted polyethylene glycols, and derivative thereof.

25. The composition of any one of the preceding numbered paragraphs, wherein the non-ionic emulsifier is caprylocaproyl polyoxyl-8 glyceride (LABRASOL®), polysorbate 40 (TWEEN® 40), polysorbate 80 (TWEEN® 80), macrogolglycerol ricinoleate (KOLLIPHOR® P188), or oleoyl polyoxyl-6 glyceride/oleoyl macrogol-6 glyceride) (LABRAFIL®).

26. The composition of any one of the preceding numbered paragraphs, wherein the concentration of non-ionic emulsifier in the composition is about 10-40 mg/ml.

27. The composition of any one of the preceding claims, wherein the mucopenetrating substance is selected from the group consisting of: bromohexine, L-cysteine methylester, bromalein, ambroxol, guaifenesin, and N-acetyl L-cysteine and dornase alfa, optionally wherein the mucopenetrating substance is bromalein or decanoic acid.

28. The composition of any one of the preceding numbered paragraphs, wherein the mucopenetrating substance is bromalein and/or the non-ionic emulsifier is oleoyl polyoxyl-6 glyceride/oleoyl macrogol-6 glyceride)(LABRAFIL®).

29. The composition of any one of the preceding numbered paragraphs, wherein the nucleic acid is deoxyribonucleic acid (DNA) or ribonucleic acid (RNA), optionally wherein the nucleic acid has a phosphorothioate backbone.

30. The composition of any one of the preceding numbered paragraphs, wherein the nucleic acid is double-stranded or single-stranded.

31. The composition of any one of the preceding numbered paragraphs, wherein the nucleic acid comprises a modification, optionally a chemical modification and/or a genetic modification.

32. The composition of any one of the preceding numbered paragraphs, wherein the nucleic acid has a length of about 10 to 50 nucleotides.

33. The composition of any one of the preceding numbered paragraphs, wherein the nucleic acid is not a locked nucleic acid (LNA) or a peptide nucleic acid (PNA).

34. The composition of any one of the preceding numbered paragraphs, wherein the therapeutic nucleic acid is selected from the group consisting of antisense oligonucleotides and RNA interference molecules.

35. The composition of any one of the preceding numbered paragraphs, wherein the RNA interference molecules are selected from the group consisting of short-hairpin RNAs (shRNAs), small-interfering RNAs (siRNAs), and micro RNAs (mRNAs).

36. The composition of any one of the preceding numbered paragraphs, wherein the therapeutic nucleic acid is an antisense oligonucleotide (ASO).

37. The composition of any one of the preceding numbered paragraphs, wherein the therapeutic nucleic acid targets SMAD7 mRNA.

38. The composition of any one of the preceding numbered paragraphs, wherein the therapeutic nucleic acid is mongersen (GED-0301).

39. The composition of any one of the preceding numbered paragraphs, wherein the cationic polymer and the therapeutic nucleic acid are present at a ratio of at least 1:1, at least 5:1, or at least 10:1 cationic polymer:therapeutic nucleic acid.

40. The composition of any one of the preceding numbered paragraphs, wherein the ASO is mongersen (GED-0301).

41. The composition of any one of the preceding numbered paragraphs, wherein the composition is a pharmaceutical composition further comprising a pharmaceutically-acceptable excipient.

42. The composition of any one of the preceding numbered paragraphs, wherein the therapeutic nucleic acid is an engineered nucleic acid, optionally a recombinant nucleic acid or a synthetic nucleic acid.

43. A cell comprising the composition of any one of the preceding numbered paragraphs.

44. A complex produced by combining a mucopenetrating substance with a therapeutic nucleic acid, and a cationic polymer in an amount sufficient to charge neutralize the therapeutic nucleic acid.

45. A method comprising delivering to a subject the composition of any one of the preceding numbered paragraphs.

46. A method comprising delivering to a subject a mucopenetrating substance, a therapeutic nucleic acid, and a cationic polymer in an amount sufficient to charge neutralize the therapeutic nucleic acid.

47. The method of paragraph 45 or 46, wherein the delivering is to a mucosal surface of the subject.

48. A method for decreasing gene expression in a subject, comprising delivering to a mucosal surface of a subject the composition of any one of the preceding numbered paragraphs, in an effective amount to decrease gene expression in a cell in a local region of the mucosal surface.

49. A method for synergistically decreasing gene expression in a subject, comprising delivering to a mucosal surface of a subject a C10 fatty acid and the composition of any one of the preceding numbered paragraphs, in an effective amount to synergistically decrease gene expression in a cell in a local region of the mucosal surface, optionally wherein the composition further comprises the C10 fatty acid.

50. The method of any one of the preceding numbered paragraphs, wherein gene expression in the subject is reduced by at least 20% relative to gene expression in a subject relative to gene expression in a subject who has not received the composition or has received a composition comprising the therapeutic nucleic acid without the cationic polymer and/or the mucopenetrating substance.

51. The method of any one of the numbered paragraphs, wherein the delivering comprises orally, rectally, or vaginally delivering.

52. The method of any one of the numbered paragraphs, wherein the composition is in a solution, is lyophilized, or is in the form of a tablet, optionally with an enteric coating.

53. The method of any one of the numbered paragraphs, wherein the mucosal surface is the gastrointestinal tract, rectal tissue, or vaginal tissue.

54. The method of any one of the numbered paragraphs, wherein the subject has a gastrointestinal disorder and/or has a compromised gastrointestinal barrier.

55. The method of any one of the numbered paragraphs, wherein the gastrointestinal disorder is an inflammatory bowel disorder, optionally irritable bowel syndrome (IBS), ulcerative colitis, or Crohn's disease.

56. The method of any one of the numbered paragraphs, wherein transport of the therapeutic nucleic acid through the mucosal surface is at least 5-fold, at least 10-fold, at least 15-fold, or at least 20-fold higher than uptake of a therapeutic nucleic acid without the cationic polymer and/or the mucopenetrating substance.

57. A multiple well plate, wherein each well of the plate comprises a receiver chamber underlying a permeable membrane onto which a mucus layer has been deposited.

58. A method for assessing mucotransport of a substance, comprising applying the substance to a well of paragraph 57, and assessing transport of the substance through the mucus layer.

59. The method of any one of the numbered paragraphs, wherein the substance comprises a mucopenetrating substance.

60. The method of any one of the numbered paragraphs, wherein the substance is a composition of any one of the preceding numbered paragraphs.

61. The composition of any one of the numbered paragraphs, wherein the cationic polymer is selected from polyallylamine (PALL), polylysine (PLL), and polyethyleneimine (PEI).

62. The composition of any one of the numbered paragraphs, wherein the cationic polymer is PALL.

63. The composition of any one of the numbered paragraphs, wherein the PALL has a molecule weight of lower than 50 kilodaltons (kDa).

64. The composition of any one of the numbered paragraphs 3, wherein the PALL has a molecular weight of about 10-20 kDa, optionally about 15 kDa.

65. The composition of any one of the numbered paragraphs, wherein the cationic polymer is PLL.

66. The composition of any one of the numbered paragraphs, wherein the PLL has a molecule weight of about 15-50 kDa.

67. The composition of any one of the numbered paragraphs, wherein the cationic polymer is PEI.

68. The composition of any one of the numbered paragraphs, wherein the PEI has a molecule weight of about 10-25 kDa.

69. The composition of any one of the numbered paragraphs, wherein the cationic polymer is branched.

70. The composition of any one of the numbered paragraphs, wherein the concentration of the cationic polymer in the composition is about 10-30 mg/ml.

71. The composition of any one of the numbered paragraphs, wherein the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

72. The composition of any one of the numbered paragraphs, wherein the concentration of the non-ionic emulsifier is about 10-40 mg/ml.

73. The composition of any one of the numbered paragraphs, wherein the cationic polymer is PALL, optionally having a molecule weight of below 50 kDa, and the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

74. The composition of any one of the numbered paragraphs, wherein the cationic polymer is PLL, optionally having a molecule weight of about 15-50 kDa, and the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

75. The composition of any one of the numbered paragraphs, wherein the cationic polymer is PEI, optionally branched PEI, optionally having a molecule weight of 10-25 kDa, and the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

76. The composition of any one of the numbered paragraphs, wherein the cationic polymer has a molecular weight of about 10-50 kDa.

77. The composition of any one of the numbered paragraphs, wherein the cationic polymer has a molecular weight of about 15-50 kDa.

78. The composition of any one of the numbered paragraphs, wherein the cationic polymer has a molecular weight of about 10-25 kDa.

79. The composition of any one of the numbered paragraphs, wherein the therapeutic nucleic acid is an antisense oligonucleotide (ASO).

81. The composition of any one of the numbered paragraphs, wherein the zwitterionic polymer is polyvinylpyrrolidine.

82. The composition of any one of the numbered paragraphs, wherein the polyvinylpyrrolidine has a molecular weight of about 50-100 kDa.

### EXAMPLES

### Example 1: Testing in Ex Vivo Pig Models Using Fluorescence In Situ Quantification

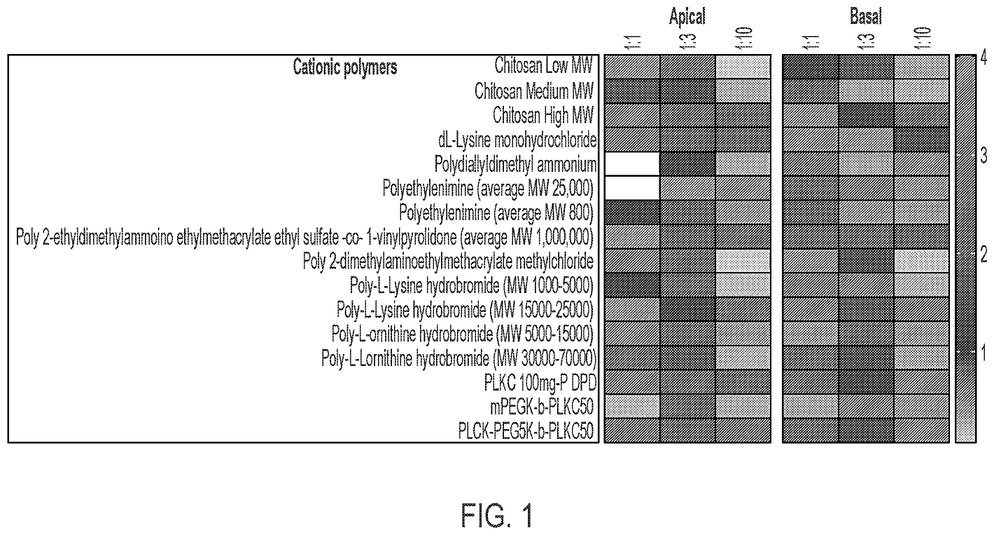

FIG. 1 shows apical and basal jejunum tissue uptake of various formulations consisting of cationic polymers at different concentrations complexed to FAM-Mongersen. Results are summarized as a heatmaps that shows fold change relative to non-formulated Mongersen. White color indicates fold changes higher than 4-fold.

Samples were prepared as follows, FAM-labelled-mongersen was dissolved in ddH2O at a concentration of 25 microM. 27 uL of cationic polymers was added at concentration of 100 mg/ml (1:1), 30 mg/ml (1:3), and 10 mg/ml (1:10) in ddH20. For tissue transport experiments, formulations were dissolved in 100 uL PBS to mimic dissolution in jejunum, mixed by pipetting and then immediately used for porcine jejunum transport studies. Transport studies were performed using a setup described earlier (https://www.nature.com/articles/s41551-020-0545-6). The samples where incubated for 1 hour, washed three times with PBS buffer followed by fluorescence intensity spectrophotometric analysis (M1000, Tecan) of the intact tissue. Experiments were performed with 4 replicates. Data was analyzed by using Prism software (Graphpad, Version 8).

Testing various cationic polymers complexed with Mongersen showed increased apical and basal tissue uptake compared to non-formulated Mongersen. The increase was both concentration dependent as well as dependent on the specific cationic polymer used.

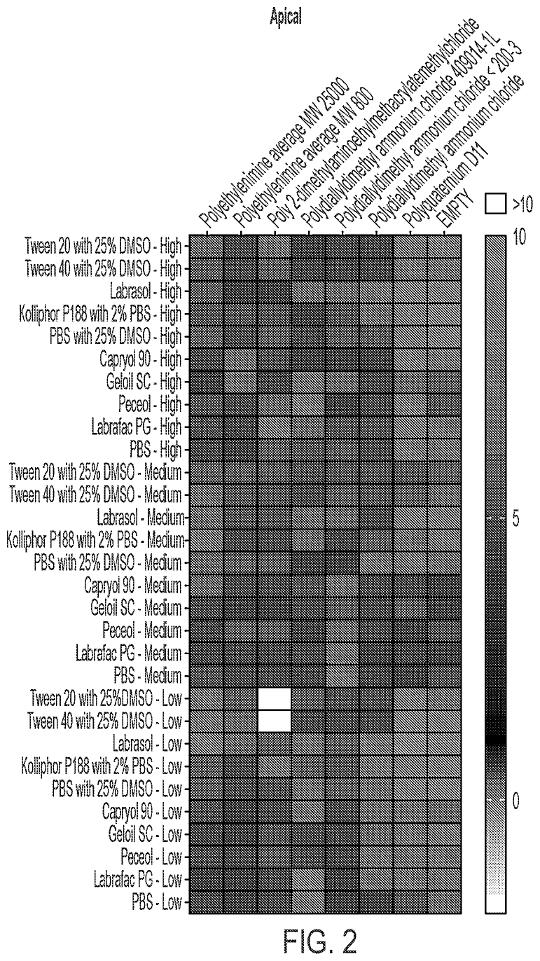

FIG. 2 shows apical (left panel) and basal (right panel) jejunum tissue uptake of various formulations consisting of cationic polymers complexed to FAM-Mongersen combined with non-ionic emulsifiers at different concentrations. Results are summarized as a heatmaps that shows fold change relative to non-formulated Mongersen. White color indicates fold changes higher than 15-fold.

Samples were prepared as follows, FAM-labelled-mongersen was dissolved in ddH2O at a concentration of 25 microM. Non-ionic emulsifiers were added at a ratio of 1:1 (10 uL of liquid emulsifier added to 10 uL 25 microM FAM-labelled-mongersen), 3:1 (30 uL of liquid emulsifier added to 10 uL 25 microM FAM-labelled-mongersen), and 5:1 (50 uL of liquid emulsifier added to 10 uL 25 microM FAM-labelled-mongersen). 27 uL of cationic polymers was added at concentration of 100 mg/ml (1:1) in ddH2O. For tissue transport experiments, formulations were dissolved in 100 uL PBS to mimic dissolution in jejunum, mixed by pipetting and then immediately used for porcine jejunum transport studies. Transport studies were performed using a setup described earlier (https://www.nature.com/articles/s41551-020-0545-6). The samples where incubated for 1 hour, washed three times with PBS buffer followed by fluorescence intensity spectrophotometric analysis (M1000, Tecan) of the intact tissue. Experiments were performed with 4 replicates. Data was analyzed by using Prism software (Graphpad, Version 8).

The results show that apical and basal tissue uptake of cationic polymer—Mongersen complexes can be further increased be the addition of non-ionic emulsifiers. The increase in fold changes is depended on both concentration and specific emulsifier and is specific to the cationic polymer used.

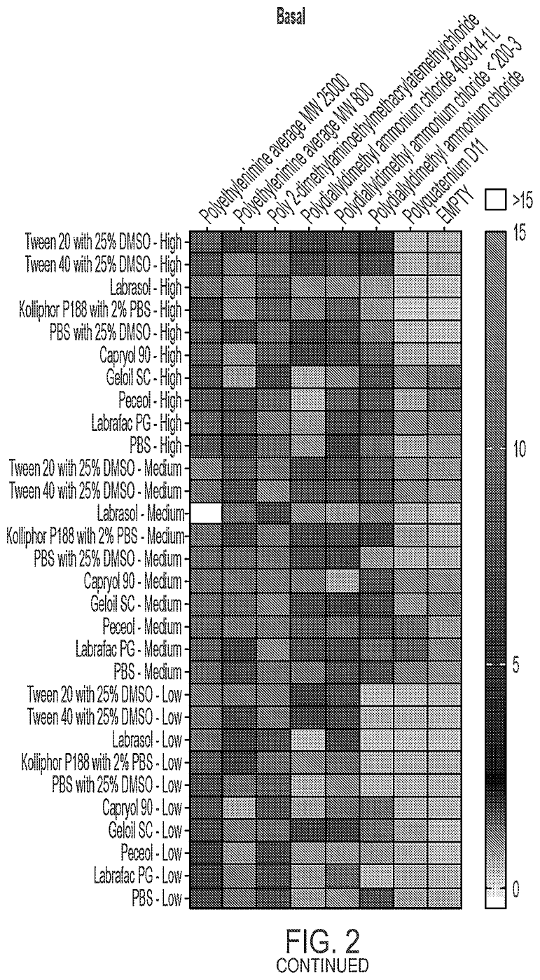

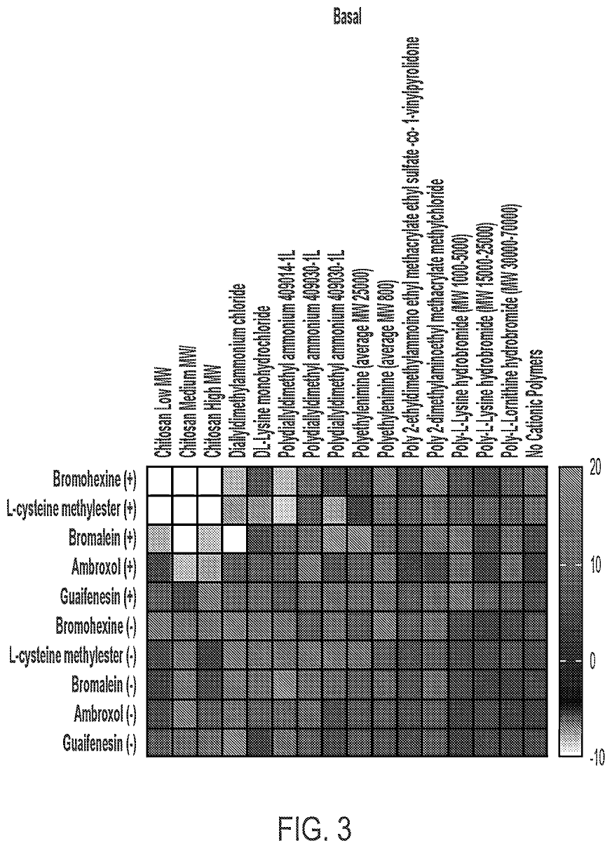

FIG. 3 shows apical and basal jejunum tissue uptake of various formulations consisting of cationic polymers complexed to FAM-Mongersen combined with mucolytic agents at different concentrations. Results are summarized as a heatmaps that shows fold change relative to non-formulated Mongersen. White color indicates fold changes higher than 20-fold.

Samples were prepared as follows, FAM-labelled-mongersen was dissolved in ddH2O at a concentration of 25 microM. Mucolytic agents were added at a ratio of 1:1 (10 uL of 100 mg/ml mucolytic agent in water added to 10 uL 25 microM FAM-labelled-mongersen). 27 uL of cationic polymers was added at concentration of 100 mg/ml (1:1) in ddH20. For tissue transport experiments, formulations were dissolved in 100 uL PBS to mimic dissolution in jejunum, mixed by pipetting and then immediately used for porcine jejunum transport studies. Transport studies were performed using a setup described earlier (https://www.nature.com/articles/s41551-020-0545-6). The samples where incubated for 1 hour, washed three times with PBS buffer followed by fluorescence intensity spectrophotometric analysis (M1000, Tecan) of the intact tissue. Experiments were performed with 4 replicates. Data was analyzed by using Prism software (Graphpad, Version 8).

The results show that apical and basal tissue uptake of cationic polymer—Mongersen complexes can be further increased be the addition mucolytic agents. The increase in fold changes is depended on both concentration and specific emulsifier and is specific to the cationic polymer used. Absolute fold increases achieved are comparable with formulations containing cationic polymer-Mongersen complex with non-ionic emulsifier.

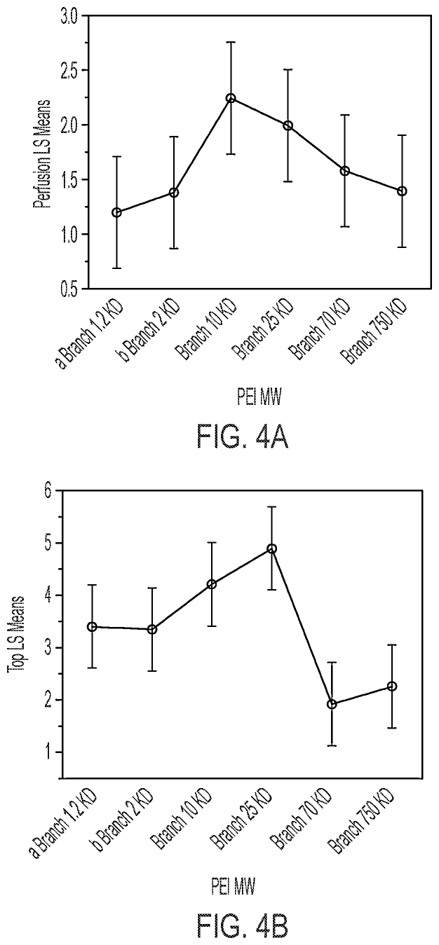

FIGS. 4A-4B show Least Squares Means Plots, which show relative change in tissue permeability and apical tissue accumulation of FAM-Mongersen using different molecular weight branched polyethyleneimine polymers. The results are based on a statistical regression analysis using 6 different non-ionic emulsifiers combined with polyethyleneimine-Mongersen polyplex.

Samples were prepared as follows, FAM-labelled-mongersen was dissolved in ddH2O at a concentration of 25 microM. Non-ionic emulsifiers (Kolliphor P188, Labrafil, Tween 20, Tween 40, Tween 60, Tween 80) were added at a ratio of 1:1 (10 uL of liquid emulsifier added to 10 uL 25 microM FAM-labelled-mongersen). 27 uL of cationic polymers was added at concentration of 100 mg/ml (1:1) in ddH20. For tissue transport experiments, formulations were dissolved in 100 uL PBS to mimic dissolution in jejunum, mixed by pipetting and then immediately used for porcine jejunum transport studies. Transport studies were performed using a setup described earlier (https://www.nature.com/articles/s41551-020-0545-6). The samples where incubated for 1 hour, washed three times with PBS buffer followed by fluorescence intensity spectrophotometric analysis (M1000, Tecan) of the intact tissue. Experiments were performed with 4 replicates. Data was analyzed by using JASP software.

The results show that apical tissue uptake and also transport across the tissue using PEI polymer—Mongersen complexes with non-ionic emulsifier is dependent on the molecular weight of PEI. The molecular weight that is showing the highest increase in tissue uptake as well as permeation is PEI with an average molecular weight of 10-25 kDa. This trend is true across all different non-ionic emulsifier combinations tested.

### Example 2: Testing in Ex Vivo Pig Models Using Immunohistochemistry (IHC) of Biopsy Samples

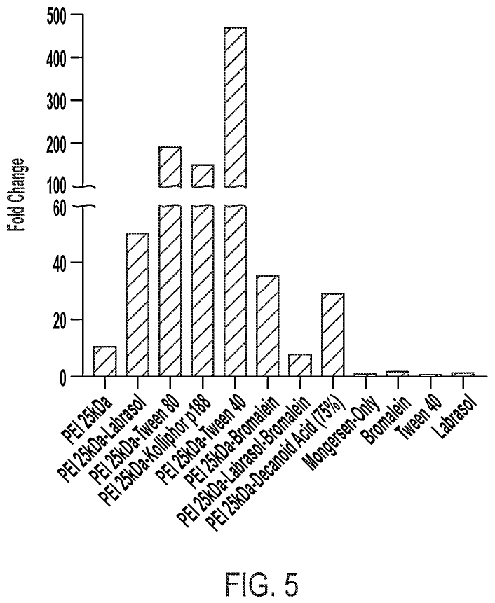

FIG. 5 shows apical and basal jejunum tissue uptake of various formulations consisting of cationic polymers complexed to FAM-Mongersen combined with additional excipients: Non-ionic emulsifiers (Kolliphor P188, Labrafil, Tween 40, Tween 80), mycolytic (bromalein) or permeability enhancer/mucodisruptor (decanoic acid). FAM fluorescence intensity was quantified by fluorescence microscopy analysis of tissue cross-sections. The results show fold change relative to non-formulated Mongersen.

Samples were prepared as follows, FAM-labelled-mongersen was dissolved in ddH2O at a concentration of 25 microM. Non-ionic emulsifiers (Kolliphor P188, Labrafil, Tween 40, Tween 80), mycolytic (bromalein) or permeability enhancer/mucodisruptor (decanoic acid) were added at a ratio of 1:1 (10 uL of liquid emulsifier added to 10 uL 25 microM FAM-labelled-mongersen). 27 uL of cationic polymers was added at concentration of 100 mg/ml (1:1) in ddH20. For tissue transport experiments, formulations were dissolved in 100 uL PBS to mimic dissolution in jejunum, mixed by pipetting and then immediately used for porcine jejunum transport studies. Transport studies were performed using a setup described earlier (https://www.nature.com/articles/s41551-020-0545-6). Biopsy samples were harvested after tissue was incubated for 1 hour and washed three times with PBS buffer followed by cyrosectioning and fluorescence microscopy analysis of cryosections. The FAM fluorescence intensity of cyrosections at a magnification of 4×was quantified by ImageJ.

The results show increased transport of FAM-Mongersen into intestinal tissue of the PEI polyplexes tested compared to non-formulated FAM Mongersen. Quantification of fluorescence intensity based on cross-sections of jejunum tissue exposed to the formulations enables to select for fluorescence signal within the GI tissue as opposed to signal outside the tissue trapped in the mucus layer. Addition of additional excipients: Non-ionic emulsifier, mycolytic (bromalein) or permeability enhancer/mucodisruptor (decanoic acid) increased tissue uptake compared to PEI polyplexes alone while the additional components were found to show no improvement when formulated without the cationic agent PEI with FAM-Mongersen. This indicates a synergistic effect of the additional excipients in transport enhancement of FAM-Mongersen.

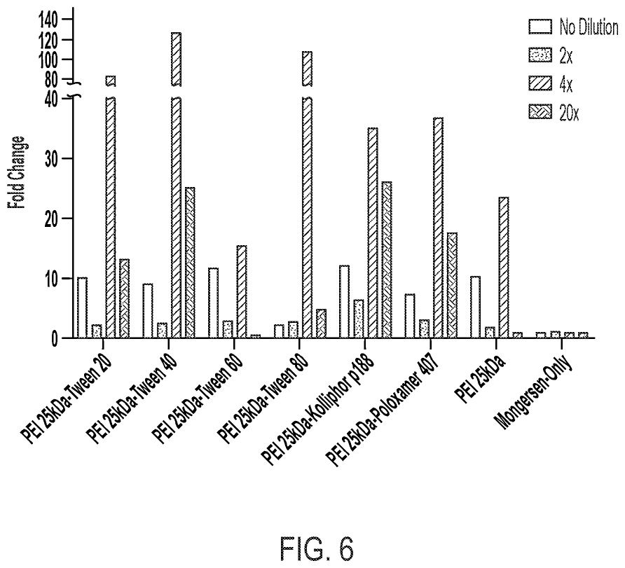

FIG. 6 shows apical and basal jejunum tissue uptake of various formulations consisting of cationic polymers complexed to FAM-Mongersen combined with non-ionic emulsifiers including Kolliphor P188, Poloxamer 407, Labrafil, Tween 20, Tween 40, Tween 60, Tween 80. FAM fluorescence intensity was quantified by fluorescence microscopy analysis of tissue cross-sections. The results show fold change relative to non-formulated Mongersen.

Samples were prepared as follows, FAM-labelled-mongersen was dissolved in ddH2O at a concentration of 25 microM. Non-ionic emulsifiers (Kolliphor P188, Labrafil, Tween 40, Tween 80), mycolytic (bromalein) or permeability enhancer/mucodisruptor (decanoic acid) were added at a ratio of 1:1 (10 uL of liquid emulsifier added to 10 uL 25 microM FAM-labelled-mongersen). 27 uL of cationic polymers was added at concentration of 100 mg/ml (1:1) in ddH20. For tissue transport experiments, formulations were dissolved in 100 uL PBS and then further diluted by the following volume ratio in simulated intestinal fluid: 1:2, 1:4, 1:20 to mimic dissolution and dilution occurring in jejunum. The formulations were mixed by pipetting and then immediately used for porcine jejunum transport studies. Transport studies were performed using a setup described earlier (https://www.nature.com/articles/s41551-020-0545-6). Biopsy samples were harvested after tissue was incubated for 1 hour and washed three times with PBS buffer followed by cyrosectioning and fluorescence microscopy analysis of cryosections. The FAM fluorescence intensity of cyrosections at a magnification of 4×was quantified by ImageJ.

The results show transport enhancement of FAM-Mongersen PEI polyplexes is retained after SIF dilution up to a factor of 4 times dilution substantially higher dilution of 20-fold results in no enhancement. This indicates that the formulation effect is dependent on the local concentration of excipients exposed to the tissue shows a broad effective concentration range.

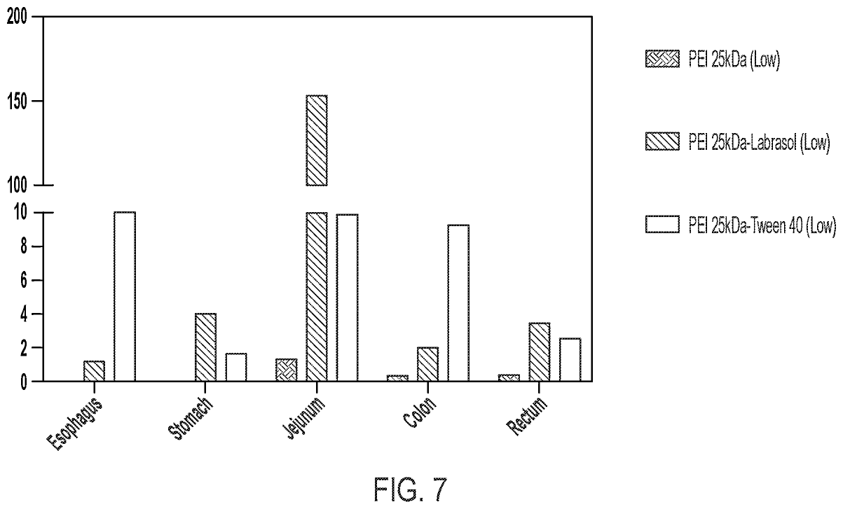

FIG. 7 shows tissue uptake in esophagus, stomach, jejunum, colon and rectum of various formulations consisting of cationic polymers complexed to FAM-Mongersen combined with non-ionic emulsifiers (Labrasol and Tween 40). FAM fluorescence intensity was quantified by fluorescence microscopy analysis of tissue cross-sections. The results show fold change relative to non-formulated Mongersen in the corresponding tissue segment.

Samples were prepared as follows, FAM-labelled-mongersen was dissolved in ddH2O at a concentration of 25 microM. Non-ionic emulsifiers (Labrasol and Tween 40), were added at a ratio of 1:1 (10 uL of liquid emulsifier added to 10 uL 25 microM FAM-labelled-mongersen). 27 uL of cationic polymers was added at concentration of 100 mg/ml (1:1) in ddH20. For tissue transport experiments, formulations were dissolved in 100 uL PBS to mimic dissolution in jejunum, mixed by pipetting and then immediately used for porcine GI tissue transport studies. Esophagus, stomach, jejunum, colon and rectum porcine tissue was used for this study based on a setup described earlier (https://www.nature.com/articles/s41551-020-0545-6). Biopsy samples were harvested after tissue was incubated for 1 hour and washed three times with PBS buffer followed by cyrosectioning and fluorescence microscopy analysis of cryosections. The FAM fluorescence intensity of cyrosections at a magnification of 4×was quantified by ImageJ.

The results show increased transport of FAM-Mongersen into intestinal tissue of the PEI polyplexes tested compared to non-formulated FAM Mongersen across the different GI tissue segments suggesting that these formulations could be effective to delivery oligonucleotides in various GI segments other than jejunum. Interestingly, depending on the formulation and tissue segment the observed fold changes change by multiple fold suggesting that each GI segment requires optimization of formulations to maximize transport.

### Example 3: Testing in In Vivo Using Pig Models and Immunohistochemistry (IHC) of Biopsy Samples

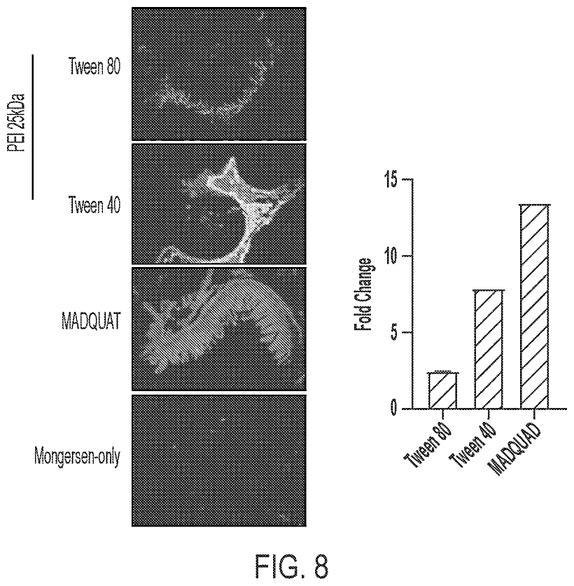

FIG. 8 shows tissue uptake of various formulations consisting of cationic polymers complexed to FAM-Mongersen combined with non-ionic emulsifiers (Labrasol and Tween 40) in jejunum. FAM fluorescence intensity was quantified by fluorescence microscopy analysis of tissue cross-sections of biopsy samples harvested from jejunum segments exposed to formulations in pigs in vivo. The results show fold change relative to non-formulated Mongersen in the corresponding tissue segment.

Samples were prepared as follows, FAM-labelled-mongersen was dissolved in ddH2O at a concentration of 25 microM. Non-ionic emulsifiers (Labrasol and Tween 40 and Tween 80), were added at a ratio of 1:1 (10 uL of liquid emulsifier added to 10 uL 25 microM FAM-labelled-mongersen). 27 uL of cationic polymers was added at concentration of 100 mg/ml (1:1) in ddH20. For tissue transport experiments, formulations were dissolved in 100 uL PBS to mimic dissolution in jejunum, mixed by pipetting and then immediately used for porcine GI tissue animal studies. For in vivo drug delivery studies female Yorkshire pigs between 50 and 80 kg in weight were used. Before every experiment, the animals were fasted overnight. On the day of the procedure the morning feed was held. The animals were sedated with an intramuscular injection of telazol (tileramine/zolazepam) 5 mg/kg, xylazine 2 mg/kg and atropine 0.04 mg/kg. After complete sedation, the small intestine was accessed surgically. 1 cm jejunum segments were created by a custom device that compresses 0.5 cm of tissue on either side by tightly controlled magnetic force. 0.57 mL of formulation was then added in these segments via needle injection. After 1 hour incubation, the fluid was removed and the exposed tissue washed three times with PBS buffer followed by cyrosectioning and fluorescence microscopy analysis of cryosections. The FAM fluorescence intensity of cyrosections at a magnification of 4×was quantified by ImageJ.

The results show increased transport of FAM-Mongersen into intestinal tissue of the PEI polyplexes tested compared to non-formulated FAM Mongersen in jejunum. This confirms the ex vivo results obtained previously and validates the predictability of the system.

### Example 4: Testing in Ex Vivo Pig Models Using Fluorescence In Situ Quantification

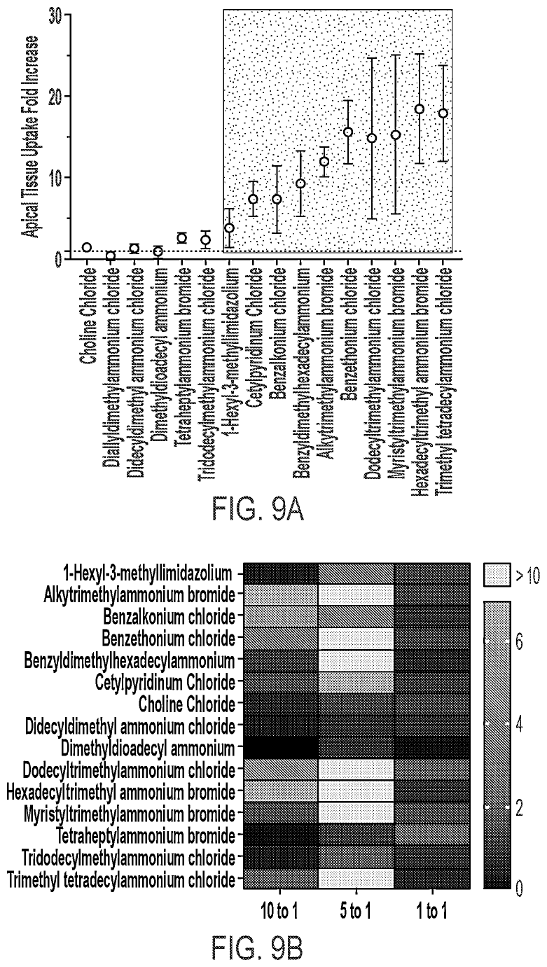

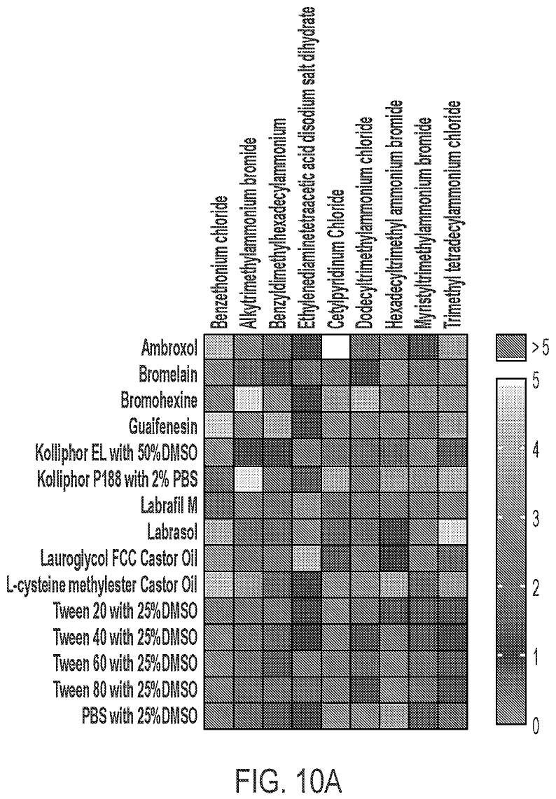

FIGS. 9A-9B show effect of charged surfactants on tissue uptake of formulations. FIG. 9A shows tissue uptake of various formulations consisting of cationic polymers complexed to FAM-Mongersen combined with non-ionic emulsifiers (Labrasol and Tween 40) in jejunum. FAM fluorescence intensity was quantified by fluorescence microscopy analysis of tissue cross-sections of biopsy samples harvested from jejunum segments exposed to formulations in pigs in vivo. FIG. 9B shows the data in FIG. 9A in the form of a heatmap as show fold change relative to non-formulated Mongersen in the corresponding tissue segment.

Samples were prepared as follows, FAM-labelled-mongersen was dissolved in ddH2O at a concentration of 25 microM. Non-ionic emulsifiers (Labrasol and Tween 40 and Tween 80), were added at a ratio of 1:1 (10 uL of liquid emulsifier added to 10 uL 25 microM FAM-labelled-mongersen). 27 uL of cationic polymers was added at concentration of 100 mg/ml (1:1) in ddH20. For tissue transport experiments, formulations were dissolved in 100 uL PBS to mimic dissolution in jejunum, mixed by pipetting and then immediately used for porcine GI tissue animal studies. For in vivo drug delivery studies female Yorkshire pigs between 50 and 80 kg in weight were used. Before every experiment, the animals were fasted overnight. On the day of the procedure the morning feed was held. The animals were sedated with an intramuscular injection of telazol (tileramine/zolazepam) 5 mg/kg, xylazine 2 mg/kg and atropine 0.04 mg/kg. After complete sedation, the small intestine was accessed surgically. 1 cm jejunum segments were created by a custom device that compresses 0.5 cm of tissue on either side by tightly controlled magnetic force. 0.57 mL of formulation was then added in these segments via needle injection. After 1 hour incubation, the fluid was removed and the exposed tissue washed three times with PBS buffer followed by cyrosectioning and fluorescence microscopy analysis of cryosections. The FAM fluorescence intensity of cyrosections at a magnification of 4×was quantified by ImageJ.

The results show increased transport of FAM-Mongersen into intestinal tissue of the PEI polyplexes tested compared to non-formulated FAM Mongersen in jejunum. This confirms the ex vivo results obtained previously and validates the predictability of the system.

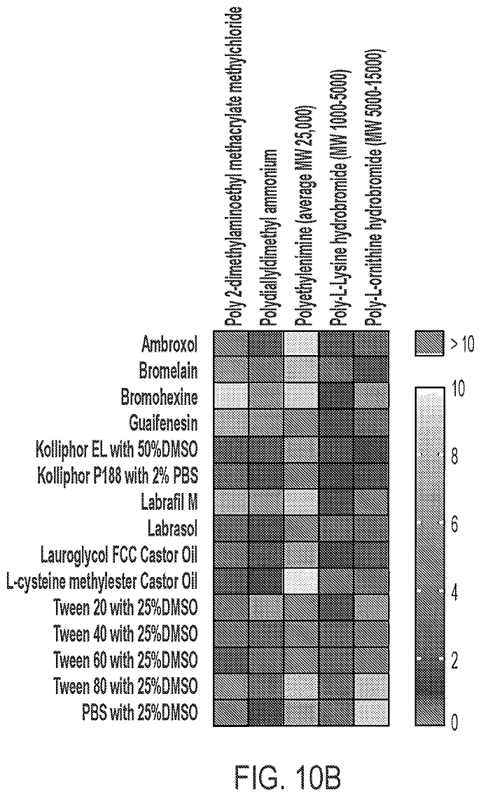

FIGS. 10A-10B show IHC analysis of the fluorescence signal of Fam labeled Mongersen in vivo in pigs in the jejunum, where the Mongersen is treated with various Mongersen-polyplex formulations. effect of charged surfactants on tissue uptake of formulations.

Samples were prepared as follows, FAM-labelled-mongersen was dissolved in ddH2O at a concentration of 25 microM. Non-ionic emulsifiers (Labrasol and Tween 40 and Tween 80), were added at a ratio of 1:1 (10 uL of liquid emulsifier added to 10 uL 25 microM FAM-labelled-mongersen). 27 uL of cationic polymers was added at concentration of 100 mg/ml (1:1) in ddH20. For tissue transport experiments, formulations were dissolved in 100 uL PBS to mimic dissolution in jejunum, mixed by pipetting and then immediately used for porcine GI tissue animal studies. For in vivo drug delivery studies female Yorkshire pigs between 50 and 80 kg in weight were used. Before every experiment, the animals were fasted overnight. On the day of the procedure the morning feed was held. The animals were sedated with an intramuscular injection of telazol (tileramine/zolazepam) 5 mg/kg, xylazine 2 mg/kg and atropine 0.04 mg/kg. After complete sedation, the small intestine was accessed surgically. 1 cm jejunum segments were created by a custom device that compresses 0.5 cm of tissue on either side by tightly controlled magnetic force. 0.57 mL of formulation was then added in these segments via needle injection. After 1 hour incubation, the fluid was removed and the exposed tissue washed three times with PBS buffer followed by cyrosectioning and fluorescence microscopy analysis of cryosections. The FAM fluorescence intensity of cyrosections at a magnification of 4×was quantified by ImageJ.

The results show increased transport of FAM-Mongersen into intestinal tissue of the PEI polyplexes tested compared to non-formulated FAM Mongersen in jejunum. This confirms the ex vivo results obtained previously and validates the predictability of the system.

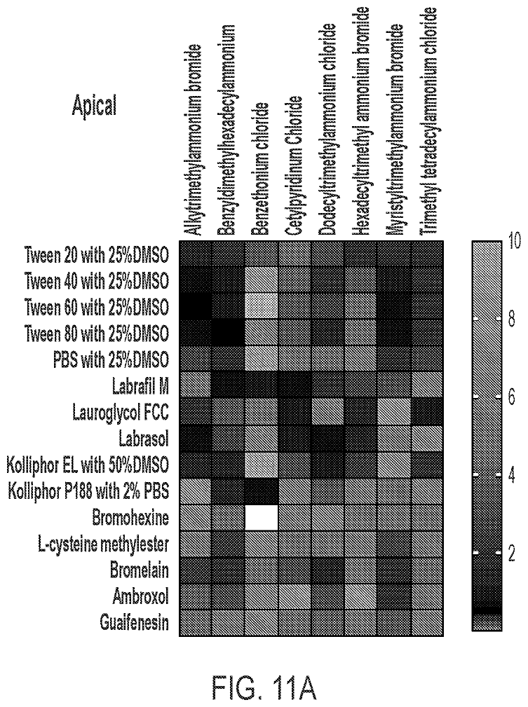

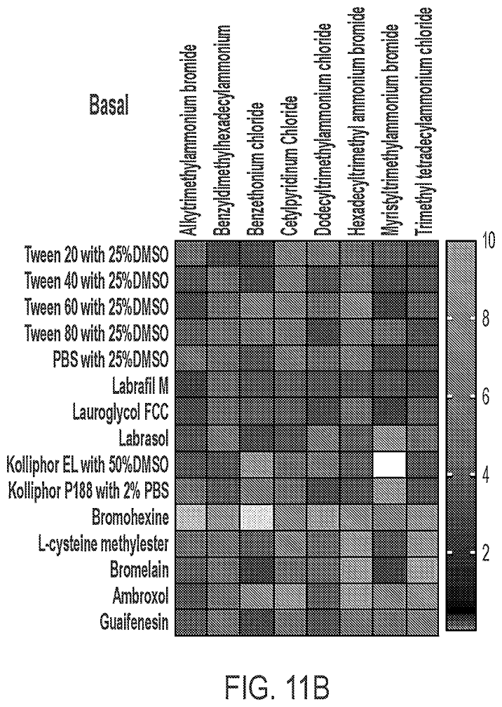

FIGS. 11A-11B shows uptake of FAM-Mongersen into apical (FIG. 11A) and basal (FIG. 11B) intestinal tissue for various formulations.

Samples were prepared as follows, FAM-labelled-mongersen was dissolved in ddH2O at a concentration of 25 microM. Non-ionic emulsifiers (Labrasol and Tween 40 and Tween 80), were added at a ratio of 1:1 (10 uL of liquid emulsifier added to 10 uL 25 microM FAM-labelled-mongersen). 27 uL of cationic polymers was added at concentration of 100 mg/ml (1:1) in ddH20. For tissue transport experiments, formulation were dissolved in 100 uL PBS to mimic dissolution in jejunum, mixed by pipetting and then immediately used for porcine GI tissue animal studies. For in vivo drug delivery studies female Yorkshire pigs between 50 and 80 kg in weight were used. Before every experiment, the animals were fasted overnight. On the day of the procedure the morning feed was held. The animals were sedated with an intramuscular injection of telazol (tileramine/zolazepam) 5 mg/kg, xylazine 2 mg/kg and atropine 0.04 mg/kg. After complete sedation, the small intestine was accessed surgically. 1 cm jejunum segments were created by a custom device that compresses 0.5 cm of tissue on either side by tightly controlled magnetic force. 0.57 mL of formulation was then added in these segments via needle injection. After 1 hour incubation, the fluid was removed and the exposed tissue washed three times with PBS buffer followed by cyrosectioning and fluorescence microscopy analysis of cryosections. The FAM fluorescence intensity of cyrosections at a magnification of 4×was quantified by ImageJ.

The results show increased transport of FAM-Mongersen into intestinal tissue of the PEI polyplexes tested compared to non-formulated FAM Mongersen in jejunum. This confirms the ex vivo results obtained previously and validates the predictability of the system.

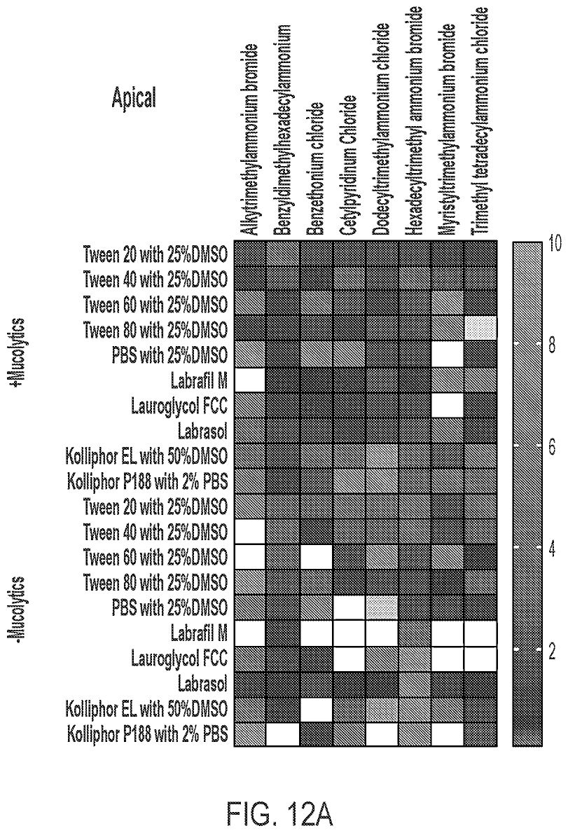

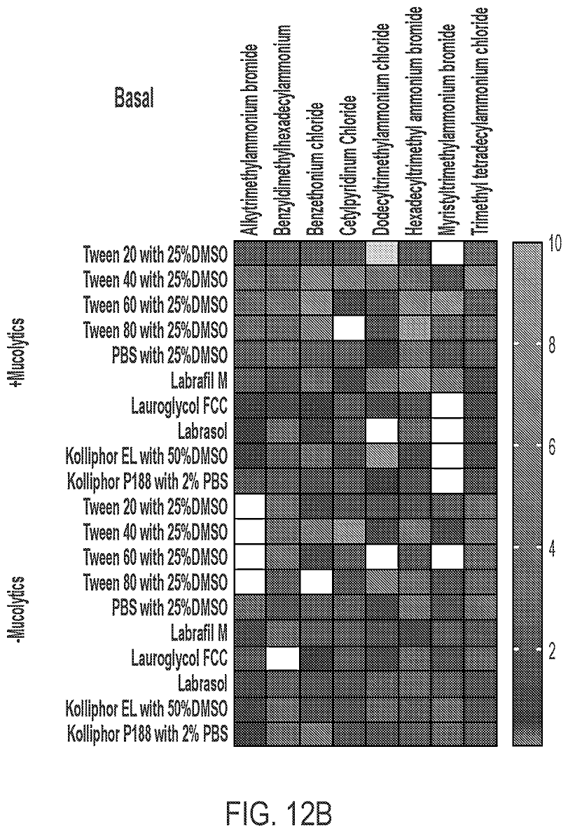

FIGS. 12A-12B shows uptake of FAM-Mongersen into apical (FIG. 11A) and basal (FIG. 11B) intestinal tissue for various formulations with and without mucolytics.

Samples were prepared as follows, FAM-labelled-mongersen was dissolved in ddH2O at a concentration of 25 microM. Non-ionic emulsifiers (Labrasol and Tween 40 and Tween 80), were added at a ratio of 1:1 (10 uL of liquid emulsifier added to 10 uL 25 microM FAM-labelled-mongersen). 27 uL of cationic polymers was added at concentration of 100 mg/ml (1:1) in ddH20. For tissue transport experiments, formulations were dissolved in 100 uL PBS to mimic dissolution in jejunum, mixed by pipetting and then immediately used for porcine GI tissue animal studies. For in vivo drug delivery studies female Yorkshire pigs between 50 and 80 kg in weight were used. Before every experiment, the animals were fasted overnight. On the day of the procedure the morning feed was held. The animals were sedated with an intramuscular injection of telazol (tileramine/zolazepam) 5 mg/kg, xylazine 2 mg/kg and atropine 0.04 mg/kg. After complete sedation, the small intestine was accessed surgically. 1 cm jejunum segments were created by a custom device that compresses 0.5 cm of tissue on either side by tightly controlled magnetic force. 0.57 mL of formulation was then added in these segments via needle injection. After 1 hour incubation, the fluid was removed and the exposed tissue washed three times with PBS buffer followed by cyrosectioning and fluorescence microscopy analysis of cryosections. The FAM fluorescence intensity of cyrosections at a magnification of 4×was quantified by ImageJ.

The results show increased transport of FAM-Mongersen into intestinal tissue of the PEI polyplexes tested compared to non-formulated FAM Mongersen in jejunum. This confirms the ex vivo results obtained previously and validates the predictability of the system.

### Example 5: Analysis of Diffusion Through Native Porcine Mucus Using TransWell System

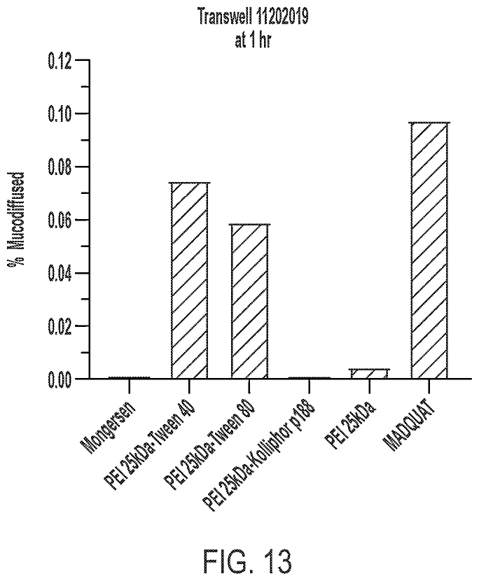

FIG. 13 shows transport through native porcine mucus obtained from the jejunum of various FAM-Mongersen formulations. Microdiffusion is calculated by measured FAM fluorescence intensity in receiver chamber compared to the initial donor fluorescence intensity after 1 hour of incubation.

Samples were prepared as follows, FAM-labelled-mongersen was dissolved in ddH2O at a concentration of 25 microM. Non-ionic emulsifiers (kolliphor P188, Tween 40 and Tween 80), were added at a ratio of 1:1 (10 uL of liquid emulsifier added to 10 uL 25 microM FAM-labelled-mongersen). 27 uL of cationic polymers was added at concentration of 100 mg/ml (1:1) in ddH20. For tissue transport experiments, formulations were dissolved in 100 uL PBS to mimic dissolution in jejunum, mixed by pipetting and then immediately used for mucus transport studies. For mucus transport studies, native mucus was collected from freshly harvested jejunum segments from adult Yorkshire pigs, and 0.3 mL was added on 24 well plate polycarbonate Transwell insert with 8 um pore size. The receiver chamber was filled with PBS buffer until contact was reached with transwell filter. Then, 0.5 mL of formulation was carefully added on each transwell filter and incubated for 1 hour. Subsequently, 50 uL of the receiver fluid was removed and FAM fluorescence intensity measured via plate reader analysis.

The results reveal that transport across the mucus layer is increased for FAM-Mongersen-PEI polyplexes in presence of a non-ionic emulsifier. FAM-Mongersen-PEI polyplexes alone show poor mucus permeability. This suggests that the non-ionic emulsifier enhance overall jejunum transport by increasing mucus penetration.

### Example 6: Gastrointestinal Tract Update with Other Oligonucleotides

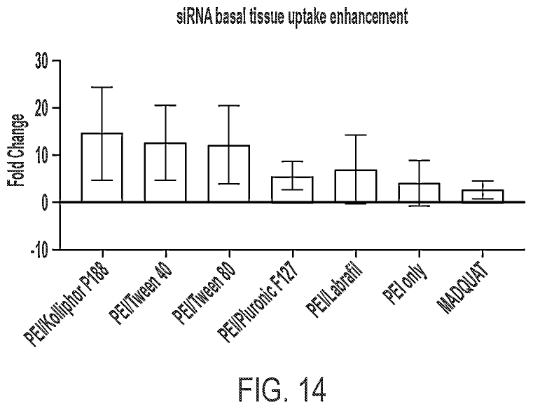

FIG. 14 shows apical jejunum tissue uptake of various formulations consisting of cationic polymers complexed to Cy5-siRNA combined with non-ionic emulsifiers. Tissue uptake was analyzed by measuring cy5 fluorescent signal intensity of ex vivo jejunum tissue exposed with Cy5-siRNA formulations for 1 hour. The results show fold change relative to non-formulated Cy5-siRNA.

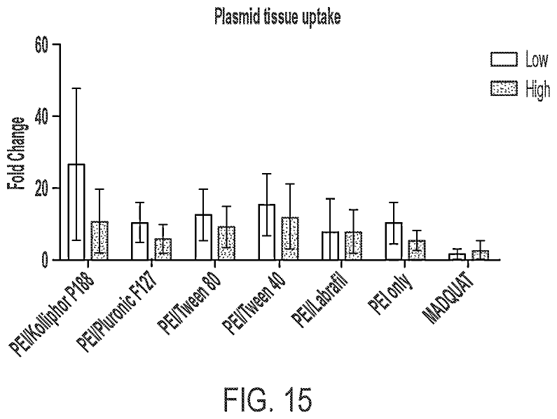

FIG. 15 shows apical jejunum tissue uptake of various formulations consisting of cationic polymers complexed to Cy3 conjugated plasmid DNA combined with non-ionic emulsifiers. Plasmid DNA was complexed with a higher and a lower concentration of cationic polymer (high, low). Tissue uptake was analyzed by measuring cy3 fluorescent signal intensity of ex vivo jejunum tissue exposed with Cy3-Plasmid DNA formulations for 1 hour. The results show fold change relative to non-formulated Cy3-Plasmid DNA.

### Example 7: GI-ORIS Studies—Fluorescence-Based Experiments Using FAM-Mongersen

The purpose of these studies was to evaluate of the effect of polymer architecture on antisense oligonucleotide (ASO) accumulation in jejunum tissue. Three (3) different cationic polymers (polyallylamine (PALL), polylysine (PLL), and polyethyleneimine (PEI)) as well as a zwitterionic polymer (polyvinylpyrrolidine (PVP)) were tested in combination with different non-ionic emulsifiers (Labrafil (LFCS), Pluronic F127, polysorbate 40 (TWEEN® 40) (T40) or polysorbate 80 (TWEEN® 80) (T80)).

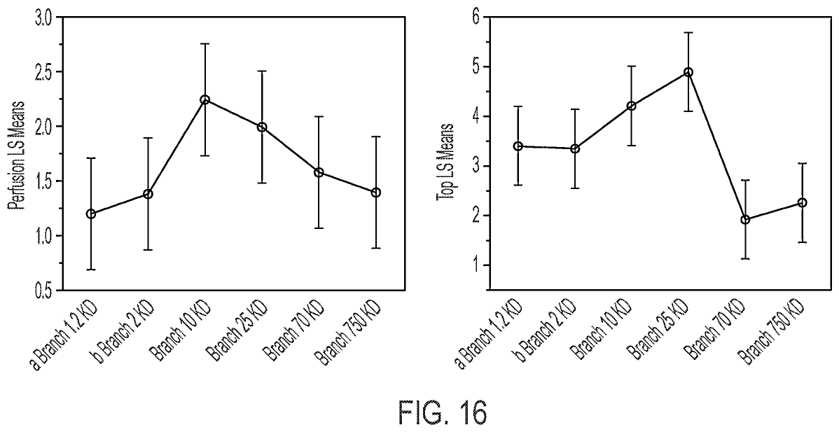

The samples were prepared with the follow concentrations of reagents: fluorescein (FAM)-labeled-Mongersen ASO in phosphate buffered saline (PBS) (300 μg/mL); cationic polymer (34 or 68 mg/mL) in PBS; non-ionic emulsifier (2.8 mg/mL) in PBS. For each study, the sample formulation was diluted by 25% to mimic dissolution in the jejunum. Transport studies were performed as described elsewhere (von Erlach T et al. Nature Biomedical Engineering volume 4, pages 544-559 (2020)). The samples were incubated for 1 hour, washed multiple times with PBS, and fluorescence intensity spectrophotometric analysis (M1000, Tec an) of the intact tissue was performed. The experiments were performed with 8 replicates. The data were analyzed using Prism software (Graphpad, Version 8) and Tableau software. Results are shown in FIGS. 16-19.

The relative change in apical tissue accumulation of FAM-Mongersen using the different molecular weight branched polyethyleneimine polymers (1.2 kilodaltons (kDa), 2 kDa, 10 kDa, 25 kDa, 70 kDa, or 750 kDa) was analyzed using a Least Squares Means Plot (FIG. 16). The results were based on a statistical regression analysis using six (6) different non-ionic emulsifiers combined with a PEI-Mongersen polyplex.

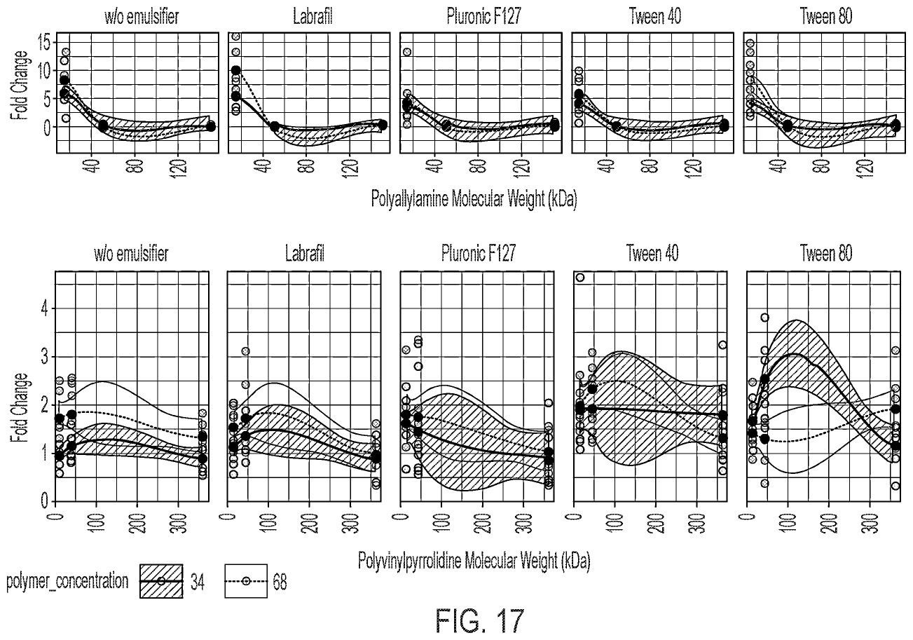

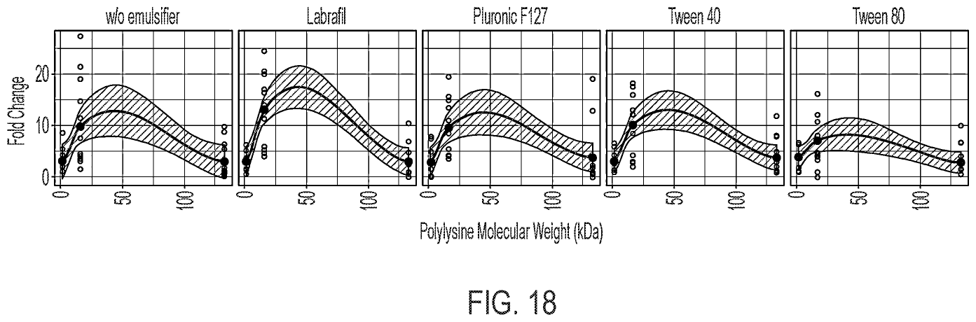

The average apical tissue accumulation of FAM-Mongersen was determined using the different molecular weight polyallylamine polymers (FIG. 17) or polylysine polymers (FIG. 18) combined with four (4) different non-ionic emulsifiers (Labrafil, Pluronic F127, polysorbate 40 (TWEEN® 40) and polysorbate 80 (TWEEN® 80). Values are expressed as fold change compared to the non-formulated FAM-labeled Mongersen control.

The most effective molecular weight ranges for each polymer and emulsifier tested are provided in Table 2.

TABLE 2
Most Effective Molecular Weights of Polymers

| Polymer | Effective Molecular Weight Range | |
|---|---|---|
| Polyethyleneimine branched | 10-25 | kDa |
| Polyallylamine | Below 50 | kDa |
| Polylysine | 16-50 | kDa |
| Polyvinylpyrrolidine | 50-100 | kDa |

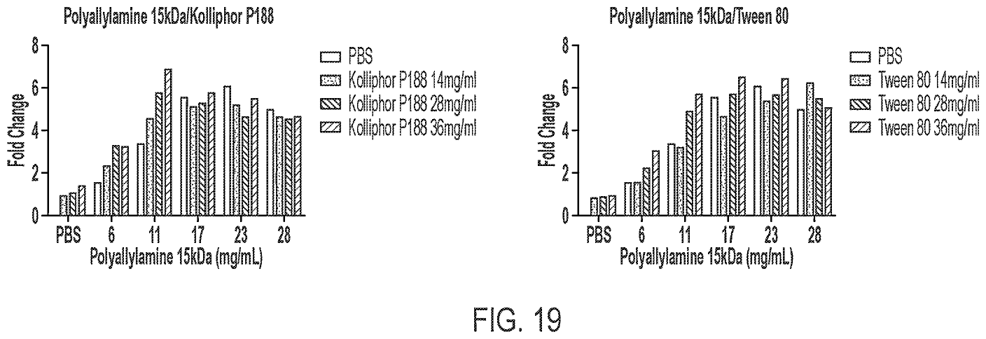

With respect to polymer and emulsifier concentration, PALL was most effective at a concentration in the range of 11-28 mg/ml regardless of emulsifier type/concentration. At that concentration, the molar ratio between PALL and FAM-Mongersen was 16 to 40. Likewise, both TWEEN® 80 and Kolliphor P188 (K188) were effective at a concentration in the range of 14-36 mg/ml. Results are summarized as a bar graph in FIG. 19, which shows fold change relative to Mongersen in PBS buffer.

### Example 8: LCMS Studies—Tissue Biopsies of the GI ORIS Tissue Plate

The purpose of these studies was to compare fluorescence signal detection of FAM-labeled Mongersen accumulation in jejunum tissue using mass spectrometry detection, relative to a non-labeled Mongersen control.

The samples were prepared with the follow concentrations of reagents: FAM-labeled Mongersen ASO in PBS (300 μg/mL); non-labeled Mongersen ASO in PBS (300 μg/mL). The Mongersen ASO was complexed to cationic polymers and emulsifiers and diluted with PBS to reach desired concentrations of the Mongersen ASO. Transport studies were performed as described elsewhere (von Erlach T et al. Nature Biomedical Engineering volume 4, pages 544-559 (2020)). The samples were incubated for 1 hour, washed multiple times with PBS, and fluorescence intensity spectrophotometric analysis (M1000, Tec an) of the intact tissue was performed. The experiments were performed with 18 replicates for fluorescence and 3 replicates for mass spectrometry. Tissue pieces were biopsied, homogenized and subsequently analyzed by mass spectrometry (Agilent 110Agilent 1100 HPLC-UV/MS0).

Non-labeled Mongersen and FAM-labeled Mongersen showed similar results in relative changes of Mongersen jejunum tissue accumulation, demonstrating that fluorescence-based detection of FAM-labeled Mongersen is a suitable method to evaluate Mongersen jejunum tissue accumulation.

Table 3 provides a summary of the results, showing average Mongersen jejunum tissue accumulation fold change of PALL 15 kDa formulations with the non-ionic emulsifiers Kolliphor P188 and TWEEN® 80, or TWEEN®80 alone, compared to non-formulated control. Fold change of non-labeled Mongersen tissue accumulation by mass spectrometry detection (LCMS) was compared to fluorescence detection using FAM-labeled Mongersen (FL). The ratio between the fold changes of the two detection methods is shown for comparison (Ratio).

TABLE 3
Summary of Results

| | Fold Change | | | Fold Change | | |
|---|---|---|---|---|---|---|
| | (relative to 160 μg/mL | | | (relative to 80 μg/mL | | |
| | Mongersen) | | | Mongersen) | | |
| Formulation | LCMS | FL | Ratio | LCMS | FL | Ratio |
| T80_28 | 1.4 | 0.9 | 1.5 | 0.7 | 0.8 | 0.9 |
| PALL: | 4.9 | 4.3 | 1.2 | 3.7 | 5.5 | 0.7 |
| 15-T80_8_28 | | | | | | |
| PALL: | 6.3 | 7.7 | 0.8 | 3.2 | 9.3 | 0.3 |
| 15-K188_17_28 | | | | | | |
| PBS | 1.0 | 1.0 | 1.0 | 1.0 | 1.0 | 1.0 |

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A composition comprising a mucopenetrating substance, a therapeutic nucleic acid, and a cationic polymer in an amount sufficient to charge neutralize the therapeutic nucleic acid.

2. The composition of claim 1, wherein the mucopenetrating substance is a non-ionic emulsifier.

3. The composition of claim 1 or 2, wherein the mucopenetrating substance has mucolytic activity.

4. The composition of any one of the preceding claims, wherein the therapeutic nucleic acid and the cationic polymer form a complex through ionic interactions.

5. The composition of claim 4, wherein the complex further comprises the mucopenetrating substance.

6. The composition of any one of the preceding claims, wherein the composition comprises at least two or at least three mucopenetrating substances.

7. The composition of any one of the preceding claims, wherein the cationic polymer is a linear polymer or a branched polymer.

8. The composition of any one of the preceding claims, wherein the cationic polymer comprises a cationic lipid.

9. The composition of any one of the preceding claims, wherein the therapeutic nucleic acid is an antisense oligonucleotide (ASO), optionally mongersen (GED-0301).

10. A composition comprising an antisense oligonucleotide (ASO), non-ionic emulsifier, and a cationic polymer, wherein the composition comprises the cationic polymer in an amount sufficient to charge neutralize the ASO.

11. The composition of any one of the preceding claims, wherein the cationic polymer is selected from polyallylamine (PALL), polylysine (PLL), and polyethyleneimine (PEI).

12. The composition of claim 11, wherein the cationic polymer is PALL.

13. The composition of claim 12, wherein the PALL has a molecule weight of lower than 50 kilodaltons (kDa).

14. The composition of claim 13, wherein the PALL has a molecular weight of about 10-20 kDa, optionally about 15 kDa.

15. The composition of claim 11, wherein the cationic polymer is PLL.

16. The composition of claim 15, wherein the PLL has a molecule weight of about 15-50 kDa.

17. The composition of claim 11, wherein the cationic polymer is PEI.

18. The composition of claim 17, wherein the PEI has a molecule weight of about 10-25 kDa.

19. The composition of claim 18, wherein the cationic polymer is branched.

20. The composition of any one of claims 10-19, wherein the concentration of the cationic polymer in the composition is about 10-30 mg/ml.

21. The composition of any one of the preceding claims, wherein the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

22. The composition of any one of any one of the preceding claims, wherein the concentration of the non-ionic emulsifier is about 10-40 mg/ml.

23. The composition of any one of any one of claim 1-10, wherein the cationic polymer is PALL, optionally having a molecule weight of below 50 kDa, and the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

24. The composition of any one of claims 1-10, wherein the cationic polymer is PLL, optionally having a molecule weight of about 15-50 kDa, and the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

25. The composition of any one of claims 1-10, wherein the cationic polymer is PEI, optionally branched PEI, optionally having a molecule weight of 10-25 kDa, and the non-ionic emulsifier is selected from oleoyl polyoxyl-6 glyceride/oleoyl macrogol-6 glyceride (LABRAFIL®), Pluronic F127, polysorbate 40 (TWEEN® 40), polysorbate 80 (TWEEN® 80), and Kolliphor P188.

26. A composition comprising a therapeutic nucleic acid, a non-ionic emulsifier, and a cationic polymer having a molecular weight of 50 kDa or lower, wherein the composition comprises the cationic polymer in an amount sufficient to charge neutralize the ASO.

27. The composition of claim 26, wherein the cationic polymer has a molecular weight of about 10-50 kDa.

28. The composition of claim 27, wherein the cationic polymer has a molecular weight of about 15-50 kDa.

29. The composition of claim 28, wherein the cationic polymer has a molecular weight of about 10-25 kDa.

30. The composition of any one of the preceding claims, wherein the cationic polymer and the therapeutic nucleic acid are present at a ratio of at least 1:1, at least 5:1, or at least 10:1 cationic polymer:therapeutic nucleic acid.

31. The composition of any one of the foregoing claims, wherein the composition is a pharmaceutical composition further comprising a pharmaceutically-acceptable excipient.

32. The composition of any one of the foregoing claims, wherein the therapeutic nucleic acid is an engineered nucleic acid, optionally a recombinant nucleic acid or a synthetic nucleic acid.

33. A cell comprising the composition of any one of the preceding claims.

34. A complex produced by combining a mucopenetrating substance, a therapeutic nucleic acid, and a cationic polymer in an amount sufficient to charge neutralize the therapeutic nucleic acid.

35. A method comprising delivering to a subject the composition of any one of the preceding claims.

36. A method comprising delivering to a subject a mucopenetrating substance, a therapeutic nucleic acid, and a cationic polymer in an amount sufficient to charge neutralize the therapeutic nucleic acid.

37. The method of claim 35 or 36, wherein the delivering is to a mucosal surface of the subject.

38. A method for decreasing gene expression in a subject, comprising delivering to a mucosal surface of a subject the composition of any one of the preceding claims, in an effective amount to decrease gene expression in a cell in a local region of the mucosal surface.

39. A method for synergistically decreasing gene expression in a subject, comprising delivering to a mucosal surface of a subject a C10 fatty acid and the composition of any one of the preceding claims, in an effective amount to synergistically decrease gene expression in a cell in a local region of the mucosal surface, optionally wherein the composition further comprises the C10 fatty acid.

40. The method of any one of the preceding claims, wherein gene expression in the subject is reduced by at least 20% relative to gene expression in a subject relative to gene expression in a subject who has not received the composition or has received a composition comprising the therapeutic nucleic acid without the cationic polymer and/or the mucopenetrating substance.

41. The method of any one of the preceding claims, wherein the mucosal surface is the gastrointestinal tract, rectal tissue, or vaginal tissue.

42. The method of any one of the preceding claims, wherein the subject has a gastrointestinal disorder and/or has a compromised gastrointestinal barrier.

43. The method of any one of the preceding claims, wherein the gastrointestinal disorder is an inflammatory bowel disorder, optionally irritable bowel syndrome (IBS), ulcerative colitis, or Crohn's disease.

44. A multiple well plate, wherein each well of the plate comprises a receiver chamber underlying a permeable membrane onto which a mucus layer has been deposited.

45. A method for assessing mucotransport of a substance, optionally a mucopenetrating substance, comprising applying the substance to a well of claim 44, and assessing transport of the substance through the mucus layer.

46. A composition comprising an antisense oligonucleotide (ASO), non-ionic emulsifier, and a zwitterionic polymer, optionally a polyvinylpyrrolidine optionally having a molecular weight of about 50-100 kDa.
